(19) United States
(12) Patent Application Publication
(10) Pub. No.: US 2014/0364775 A1
(43) Pub. Date: Dec. 11, 2014

(54) SYSTEMS AND METHODS FOR DELIVERING ULTRASONIC ENERGY TO A BODILY TISSUE

(71) Applicant: Med-Sonics Corporation, Erie, PA (US)

(72) Inventors: Shu DU, Erie, PA (US); Tao SONG, Erie, PA (US)

(73) Assignee: MED-SONICS CORPORATION, Erie, PA (US)

(21) Appl. No.: 14/299,627

(22) Filed: Jun. 9, 2014

Related U.S. Application Data
(60) Provisional application No. 61/833,154, filed on Jun. 10, 2013.

(51) Int. Cl. A61N 7/00

(52) U.S. Cl. CPC A61N 7/00
USPC 601/2

(57) ABSTRACT

A coupler includes a first portion and a second portion, and defines a passageway configured to fixedly receive a proximal end portion of a transmission member. The first portion is configured to be coupled to an ultrasonic energy source. The coupler is configured to transfer at least a portion of an ultrasonic vibration produced by the ultrasonic energy source to the transmission member. Furthermore, the first portion and the second portion are collectively configured to adjust a resonant frequency of the transmission member to correspond to a vibrational frequency of the ultrasonic vibration produced by the ultrasonic energy source. In some embodiments, the portion of the ultrasonic vibration includes a linear component. In such embodiments, the first portion and the second portion are collectively configured to transform at least a portion of the linear component of the ultrasonic vibration into a torsional component within the transmission member.

### CROSS REFERENCE TO RELATED APPLICATIONS

This application claims priority to and the benefit of U.S. Provisional Patent Application Ser. No. 61/833,154 entitled, “Systems and Methods for Delivering Ultrasonic Energy to a Bodily Tissue,” filed Jun. 10, 2013, the disclosure of which is incorporated herein by reference in its entirety.

### BACKGROUND

Embodiments described herein relate generally to systems and devices for use in conjunction with an ultrasonic ablation device and, more specifically, to coupler members configured to produce torsional vibration in the transmission members and to match the ultrasonic vibrational frequency of various transmission members with an ultrasonic energy source.

Known ultrasonic energy transmission systems are used in many different medical applications, such as, for example, for medical imaging, to disrupt obstructions and/or ablate bodily tissue. In known ultrasonic energy transmission systems for tissue ablation, ultrasonic energy is transferred from an ultrasonic energy source through a transducer horn and then a transmission member, such as a wire or tube or the like, to a distal head. Ultrasonic energy propagates through the transmission member as a periodic wave thereby causing the distal head to vibrate. Such vibrational energy can be used to ablate or otherwise disrupt bodily tissue, for example, a vascular obstruction, a kidney stone or the like. To effectively reach various sites for treatment of intravascular occlusions or regions within the kidney and urinary tract, such ultrasonic transmission members often have lengths of about 43 cm or longer.

Known ultrasonic transmission members are constructed to be flexible enough to be passed through various bodily lumens, but also with sufficient strength to transmit ultrasonic energy to the distal tip (e.g., to ablate vascular or urinary obstructions). A stronger, more durable transmission member allows for greater transmission of energy but may not be flexible or thin enough to be advanced through the vasculature to a desired treatment area. A thinner transmission member can be more flexible but is less durable and more susceptible to breakage.

Known ultrasonic energy transmission systems can be configured to deliver energy of a desired vibrational mode (e.g., waveform shape) and/or within a desired vibrational frequency range to a bodily tissue. Similarly stated, a key parameter of known ultrasonic ablation systems for efficiently ablating or otherwise disrupting a bodily tissue, for example, a vascular obstruction, a kidney stone, or the likes, is the vibrational mode and the vibrational frequency. Accordingly, known systems are often configured to produce ultrasonic energy having a frequency that matches the natural frequency of the energy delivery assembly (i.e., the transducer and/or probe assembly). When operating at the natural frequency (i.e., at resonance conditions), the amplitude of the ultrasonic energy wave (or signal) travelling through the transmission member is at its maximum. The transmission member can be thought of as having a standing wave of ultrasonic energy traveling along its length. More particularly, the standing wave produces a series of nodes (regions of minimum displacement) and anti-nodes (regions of maximum displacement) along the length of the transmission member. Thus, when operating in resonance conditions, the displacement and/or vibration at the anti-nodes are at a maximum for a given power level. Each of the anti-nodes can produce cavitations in fluids in contact with the transmission member to cause the destruction of the adjacent tissue.

Some known ultrasonic ablation systems are configured to operate at a high vibrational frequency (e.g. 25 kHz or higher) to produce higher momentum. Additionally, current standards, such as the International Standard IEC-61847, limit operating ultrasonic devices for tissue disruption at vibrational frequencies of below 20 kHz, as this is the threshold of human audible range. Accordingly, some known ultrasonic ablation systems are configured to operate at frequencies of 25 kHz or higher to ensure that variations in the system will not result in operating below 20 kHz.

A higher frequency can be, however, associated with higher heat generation in a transducer assembly of the system. Furthermore, when operating at frequencies above 25 kHz and/or 28 kHz, the mechanical system and components included in the transducer assembly may be unable to respond to the electronic signals. This can lead to a reduction in the displacement of a tip of the transmission member in contact with a portion of the bodily tissue. It would therefore be beneficial to have an ultrasonic ablation system that can operate in a tight ultrasonic vibrational frequency range of slightly above 20 kHz, for example, in the range of about 20 kHz to about 21 kHz.

Maintaining of ultrasonic vibrational frequency in this tight range requires that the vibrational characteristics and/or natural frequencies of the transmission components or assemblies included in the ultrasonic ablation system are matched. This can be difficult, particularly when transmission members of different mass, rigidity, length and/or cross section (e.g., rigid, flexible, or semi-flexible) are used. In particular, changes to the components of known ultrasonic energy transmission systems, such as a transducer assembly and a transmission member, can significantly change the system natural frequency, and thus the frequency of the delivered vibrational mode. For example, known transmission members can be configured to have varying degrees of flexibility, varying lengths and the like to accommodate to the needs of a subject. Changes in such characteristics, however, can result in transmission members that have significantly different resonant frequencies of vibration. To ensure that the desired vibrational frequencies are delivered by the ultrasonic energy transmission system, the vibrational frequencies of the transmission member and the transducer assembly should be matched.

One option is to design and fabricate transmission members to have a natural frequency that matches the desired vibrational frequencies and/or the natural frequency of the transducer assembly. However, the size and flexibility of transmission members is constrained by anatomical considerations and location of the bodily tissue, which leaves little room for adjustments to the vibrational frequency of the transmission member. Another option is to use a separate transducer assembly and/or ultrasonic horn that is matched to the desired transmission member. Designing a separate transducer assembly for each transmission member however is tedious and can significantly add to the cost of the system.

Furthermore, conventional ultrasonic ablation systems are configured to produce a linear ultrasonic vibration in the transmission members. In some applications such as intravascular ultrasonic ablation therapeutic procedures, the linear ultrasonic vibration may not be sufficient to efficiently perform the procedure (e.g., ablating a clot, cancer cell, etc.). Other modes or otherwise components of the ultrasonic vibration, for example, a torsional component, a bending component, or any combination of the linear, torsional, and/or bending component can yield better results. However, conventional devices do not allow production of any other component of the vibration thereby, limiting the ultrasonic ablation therapy to solely the linear component of the ultrasonic vibration.

Thus, a need exists for improved systems, devices, and methods for ultrasonic ablation therapy.

### SUMMARY

Embodiments described herein relate generally to systems and devices for use in conjunction with an ultrasonic ablation device and, more specifically, to coupler members configured to produce torsional vibration in the transmission members and/or to match the ultrasonic vibrational frequency of various transmission members with an ultrasonic energy source. In some embodiments, the apparatus includes a coupler including a first portion and a second portion. The coupler defines a passageway configured to fixedly receive a proximal end portion of a transmission member. The first portion is configured to be coupled to an ultrasonic energy source. The coupler is configured to transfer at least a portion of an ultrasonic vibration produced by the ultrasonic energy source to the transmission member. Furthermore, the first portion and the second portion are collectively configured to adjust a resonant frequency of the transmission member to correspond to a vibrational frequency of the ultrasonic vibration produced by the ultrasonic energy source. In some embodiments, the portion of the ultrasonic vibration includes a linear component. In such embodiments, the first portion and the second portion are collectively configured to transform at least a portion of the linear component of the ultrasonic vibration into a torsional component within the transmission member.

### BRIEF DESCRIPTION OF THE DRAWINGS

FIG. 1 is an illustration of a system for delivering ultrasonic energy to a bodily tissue according to an embodiment.

FIG. 2 is a cross-sectional view of an ultrasonic transducer assembly included in the system of FIG. 1.

FIG. 3 is a schematic illustration of a transmission member, according to an embodiment.

FIG. 4 is an enlarged view of a portion of a probe assembly according to an embodiment coupled to a transducer horn.

FIG. 5A is a perspective view of a coupler according to an embodiment. FIG. 5B is a cross section of the coupler shown in FIG. 5A taken along line A-A.

FIG. 6A is a perspective view of a coupler according to an embodiment. FIG. 6B is a cross section of the coupler shown in FIG. 6A taken along line B-B.

FIG. 7A is a perspective view of a coupler according to an embodiment. FIG. 7B is a cross section of the coupler shown in FIG. 7A taken along line C-C.

FIG. 8 is a cross section of a coupler according to an embodiment.

FIG. 9 is a cross section of a coupler according to an embodiment.

FIG. 10 is a cross section of a coupler according to an embodiment.

FIG. 11 is a cross section of a coupler according to an embodiment.

FIG. 12 is a perspective view of a transducer assembly for receiving a flexible transmission member or a semi-flexible transmission member, according to an embodiment.

FIG. 13 is a perspective view of a transducer assembly for receiving a rigid transmission member according to an embodiment.

FIG. 14 is an exploded view of an ultrasonic generator included in an ultrasonic ablation system, according to an embodiment.

FIG. 15 is a flow diagram illustrating a method for delivering a torsional component of an ultrasonic vibration using a transmission member coupled to an ultrasonic energy source.

FIG. 16 is a flow diagram illustrating a method for determining if a transmission member has a first flexural stiffness or a second flexural stiffness from identification of its resonant frequency, according to an embodiment.

### DETAILED DESCRIPTION

Embodiments described herein relate generally to systems and devices for use in conjunction with an ultrasonic ablation device and, more specifically, to couplers configured to adjust and/or match the ultrasonic vibrational frequency of a transmission member with that of an ultrasonic energy source. In some embodiments, the apparatus includes a coupler including a first portion and a second portion. The coupler defines a passageway configured to fixedly receive a proximal end portion of a transmission member. The first portion is configured to be coupled to an ultrasonic energy source. The coupler is configured to transfer at least a portion of an ultrasonic vibration produced by the ultrasonic energy source to the transmission member. Furthermore, the first portion and the second portion are collectively configured to adjust a resonant frequency of the transmission member to correspond to a vibrational frequency of the ultrasonic vibration produced by the ultrasonic energy source. In some embodiments, the portion of the ultrasonic vibration includes a linear component. In such embodiments, the first portion and the second portion are collectively configured to transform at least a portion of the linear component of the ultrasonic vibration into a torsional component within the transmission member.

In some embodiments, ultrasonic ablation systems, devices and methods described herein include unique features and components that, when coupled together, are operative to generate a vibrational frequency in a range of between about 20 kHz and about 21 kHz, for example, about 20.1 kHz, 20.2 kHz, 20.3 kHz, 20.4 kHz, 20.5 kHz, 20.6 kHz, 20.7 kHz, 20.8 kHz, or about 20.9 kHz, inclusive of all ranges and values therebetween. Embodiments described herein can include couplers configured to couple a transmission member with a transducer assembly. Each of the couplers described herein is coupled to a transmission member having a different flexibility, for example, rigid, semi-flexible or flexible transmission members. Each of the multiple couplers defines a shape and size, mass and/or a feature configured to maintain and/or adjust a natural frequency of the transmission member in the range of between about 20 kHz and about 21 kHz. In this manner, the system can operate within a tightly-controlled frequency range irrespective of the transmission member coupled to the transducer assembly. Furthermore, embodiments of the couplers described herein can also be configured to transform at least a portion of a linear component of an ultrasonic vibration provided by an ultrasonic energy source into a torsional component within the transmission member. Embodiments described herein therefore provide several advantages, including allowing the operation of an ultrasonic vibrational system at a desired frequency range of between about 20 kHz and about 21 kHz, for example, about 20.1 kHz, 20.2 kHz, 20.3 kHz, 20.4 kHz, 20.5 kHz, 20.6 kHz, 20.7 kHz, 20.8 kHz, or about 20.9 kHz, inclusive of all ranges and values therebetween. Such operation provides several advantages including, for example: (1) reducing or otherwise minimizing heat generation (by operating at a lower frequency); (2) reducing or otherwise minimizing ultrasonic vibrational frequency losses due to mismatched vibrational mode and frequencies; (3) increasing or otherwise maximizing tip displacement of transmission member (i.e., improve the efficiency of the system); and/or (4) transforming at least a portion of linear component of an ultrasonic vibration into a torsional component thereby, providing more effective ultrasonic vibration.

In some embodiments, the apparatus includes a coupler including a first portion and a second portion. The coupler defines a passageway configured to fixedly receive a proximal end portion of a transmission member. The first portion is configured to be coupled to an ultrasonic energy source. The coupler is configured to transfer at least a portion of an ultrasonic vibration produced by the ultrasonic energy source to the transmission member. Furthermore, the first portion and the second portion are collectively configured to adjust a resonant frequency of the transmission member to correspond to a vibrational frequency of the ultrasonic vibration produced by the ultrasonic energy source. In some embodiments, the first portion has a first diameter and a first length, and the second portion has a second diameter and a second length, the first diameter greater than the second diameter. The ratio of the first length and the second length can be such that the resonant frequency of the transmission member is in the range of about 20 kHz to about 21 kHz. In some embodiments, the portion of the ultrasonic vibration includes a linear component. In such embodiments, the first portion and the second portion are collectively configured to transform at least a portion of the linear component of the ultrasonic vibration into a torsional component within the transmission member.

In some embodiments, an apparatus includes a coupler including a first portion and a second portion. The coupler defines a passageway configured to fixedly receive a proximal end portion of a transmission member. The first portion is configured to be coupled to an ultrasonic energy source. The coupler is configured to transfer at least a portion of an ultrasonic vibration produced by the ultrasonic energy source to the transmission member. The portion of the ultrasonic vibration includes a linear component. The first portion and the second portion are collectively configured to transform at least a portion of the linear component of the ultrasonic vibration into a torsional component within the transmission member. In some embodiments, an outer surface of the first portion is discontinuous from an outer surface of the second portion. In some embodiments, the coupler includes a third portion disposed between the first portion and the second portion. The third portion defines a groove, such that the first portion, the second portion and the third portion are collectively configured to produce the torsional component within the transmission member.

In some embodiments, a kit includes a first transmission member and a second transmission member. A proximal end portion of the first transmission member is fixedly coupled to a first coupler. The first coupler defines a passageway in fluid communication with an irrigation lumen of the first transmission member. The first coupler is configured to couple the first transmission member to an ultrasonic transducer assembly to transfer at least a portion of a first ultrasonic vibration from the ultrasonic transducer assembly to the first transmission member. The first coupler is configured to such that the first transmission member and the first coupler have a first resonant frequency. A proximal end portion of the second transmission member is fixedly coupled to a second coupler. The second coupler defines a passageway in fluid communication with an irrigation lumen of the second transmission member. The second coupler is configured to couple the second transmission member to the ultrasonic transducer assembly to transfer at least a portion of a second ultrasonic vibration from the ultrasonic transducer assembly to the second transmission member. The second coupler is configured to such that the second transmission member and the second coupler have a second resonant frequency, different than the first resonant frequency. In some embodiments, the first transmission member defines a first flexural stiffness, and the second transmission member defines a second flexural stiffness different than the first flexural stiffness. In some embodiments, the ultrasonic transducer assembly including a control module configured to detect the first resonant frequency and the second resonant frequency. The control module is configured to produce a signal associated with at least one of the first transmission member when the first transmission member is coupled to the ultrasonic transducer assembly or the second transmission member when the second transmission member is coupled to the ultrasonic transducer assembly

In some embodiments, a method includes coupling a transmission member to an ultrasonic energy source via a coupler. A proximal end portion of the transmission member is fixedly coupled to the coupler. A distal end portion of the transmission member is inserted into a bodily lumen. A linear ultrasonic vibration is transmitted from the ultrasonic energy source to the transmission member. At least a portion of the linear ultrasonic vibration is transformed into a torsional ultrasonic vibration within the transmission member. In some embodiments, the coupler includes a first portion, a second portion, and a third portion defining a groove. The first portion, the second portion, and the third portion are collectively configured to transform at least a portion of the linear ultrasonic vibration into the torsional ultrasonic vibration within the transmission member.

In some embodiments, a method includes detecting a resonant frequency of the transmission member. In such embodiments, a signal is produced associated with the resonant frequency of the transmission member and used to determine if the transmission member has (a) a first flexural stiffness or (b) a second flexural stiffness.

As used in this specification, the terms “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

As used herein, the terms “about” and “approximately” generally mean plus or minus 10% of the value stated. For example, about 0.5 would include 0.45 and 0.55, about 10 would include 9 to 11, about 1000 would include 900 to 1100.

As used herein, the term “set” can refer to multiple features or a singular feature with multiple parts. For example, when referring to set of walls, the set of walls can be considered as one wall with multiple portions, or the set of walls can be considered as multiple, distinct walls. Thus, a monolithically-constructed item can include a set of walls. Such a set of walls can include, for example, multiple portions that are either continuous or discontinuous from each other. A set of walls can also be fabricated from multiple items that are produced separately and are later joined together (e.g., via a weld, an adhesive, or any suitable method).

As used herein, the term “target tissue” refers to an internal or external tissue of or within a patient to which ultrasonic energy ablation techniques are applied. For example, a target tissue can be cancer cells, tumor cells, lesions, vascular occlusions, thrombosis, calculi, uterine fibroids, bone metastases, adenomyosis, kidney stone, or any other bodily tissue. Furthermore, the presented examples, of target tissues are not an exhaustive list of suitable target tissues. Thus, the ultrasonic energy systems described herein are not limited to the treatment of the aforementioned tissues and can be used on any suitable bodily tissue. Moreover, a “target tissue” can also include an artificial substance within or associated with a body, such as for example, a stent, a portion of an artificial tube, a fastener within the body or the like. Thus, for example, the ultrasonic energy systems described herein can be used on or within a stent or artificial bypass graft.

As used herein, the term “stiffness” relates to an object's resistance to deflection, deformation, and/or displacement produced by an applied force, and is generally understood to be the opposite of the object's “flexibility.” For example, a wall of a tube with greater stiffness is more resistant to deflection, deformation and/or displacement when exposed to a force than a wall of a tube having a lower stiffness. Similarly stated, a tube having a higher stiffness can be characterized as being more rigid than a tube having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of the portion of the object different than the portion of the object to which the force is directly applied. Said another way, in some objects, the point of deflection is distinct from the point where the force is applied.

Stiffness (and therefore, flexibility) is an extensive property of the object being described, and thus is dependent upon the material from which the object is formed as well as certain physical characteristics of the object (e.g., cross-sectional shape, length, boundary conditions, etc.). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness of the object can be decreased by decreasing and/or changing the shape of the object.

The stiffness (or inversely, the flexibility) of an elongated object, such as a catheter or tube can be characterized by its flexural stiffness. The flexural stiffness of an object can be used to characterize the ease with which the object deflects under a given force (e.g., the ease with which the object deflects when the object is moved along a tortuous path within the body). The flexural stiffness of an object, such as a catheter, transmission member or the like, can be mathematically expressed as shown below:

k=3EI/L³

where k is the flexural stiffness of the object, E is the modulus of elasticity of the material from which the object is constructed, I is the area moment of inertia of the object (defined below), and L is the length of the object. Therefore, a “rigid” object can have a flexural stiffness such that it does not demonstrate any substantial deflection, deformation, or otherwise displacement when exposed to a first force. On the contrary, a “flexible” object can have a flexural stiffness such that it substantially deflects, deforms, or otherwise displaces when exposed to the first force. Thereby, a “semi-flexible” or “semi-rigid” object can have an intermediate flexural stiffness relative to the rigid object and the flexible object.

As used herein, the terms “cross-sectional area moment of inertia,” “area moment of inertia,” and/or “second moment of area” relate to an object's resistance to deflection or displacement around an axis that lies in a cross-sectional plane. The area moment of inertia is dependent on the cross-sectional area and/or shape of the object and can be mathematically expressed as a function of a cross-section of the object. The area moment of inertia of an object (e.g., such as the tubes disclosed herein) is described having units of length to the fourth power (e.g., in⁴, mm⁴, cm⁴, etc.). In this manner, the “area moment of inertia” is differentiated from the “moment of inertia” or “mass moment of inertia” which is expressed having units of mass times units of length to the second power (e.g., kg*m², lbₘ*ft², etc.).

Two mathematical formulas are used herein to define an area moment of inertia for a substantially annular cross-sectional shape and for a substantially arc-shaped cross-sectional shape. The area moment of inertia for the annular cross-section shape is expressed below as:

$$I = \frac{π ( {d}_{o}^{4} - {d}_{i}^{4} )}{64}$$

where dₒ is an outside diameter of the annulus and dᵢ is an inner diameter of the annulus.

The area moment of inertia for an arced cross-sectional shape is expressed below as:

$$I = \frac{{r}^{3} t}{2} [ α + cos ( α - \frac{π}{2} ) ]$$

where r is the radius of the arc, t is the thickness of the arc segment (e.g., dₒ−dᵢ), and α is the subtended angle of the radius. For continuity with the area moment of inertia equation for the annular cross-section, the equation can be expressed as shown below:

$$I = \frac{{d}_{i}^{3} ( {d}_{o} - {d}_{i} )}{16} [ α + cos ( α - \frac{π}{2} ) ]$$

As used herein, the term “working length” refers to the operational length of an object. For example, the length of an ultrasonic probe that can be communicated within an internal cavity of a subject, for example, the urinary tract is considered as the working length.

As used herein, the terms “linear component” and “torsional component” with reference to an ultrasonic vibration, is used to refer to the linear mode and the torsional mode of the ultrasonic vibration. Vibration is a mechanical phenomenon whereby oscillations occur about an equilibrium point. An object experiencing vibration (or through which vibratory energy is passed) can have many degrees of freedom (or be constrained in a manner) such that the vibration can occur in any direction along or about the axes of the object, which are known as the mode shapes or components of vibration. The most common components of vibration include: (i) linear component or mode shape which refers to vibration along a linear axis of an object; (ii) torsional component or mode shape which refers to a rotational vibration about a linear axis of the object; and (iii) bending component or mode shape which refers to a bending vibration about a linear axis of the object. The object can experience or produce a single component of the vibration or a combination thereof, for example, a combination of a linear component and a torsional component. Where an object is characterized by multiple degrees of freedom, the components of vibration are generally represented by their Eigen values and Eigen vectors, as are commonly known in the arts, according to the following equation:

{xₙ}=q₁{Ψ}₁+q₂{Ψ}₂+ . . . +qₙ{Ψ}ₙ

Where {xₙ} is a matrix representing vibrations (or displacements) in n directions, i.e., all possible components or mode shapes of vibration, q is an Eigen value and {Ψ} is an Eigen vector corresponding to a particular component or otherwise mode shape of vibration. Thus, an object (e.g., any of the transmission members described herein) can have multiple components or mode shapes of vibration when energy is conveyed therethrough. For example, the transmission member can include a combination of a linear component characterized by a first Eigen value and a first Eigen vector, and a torsional component characterized by a second Eigen value and a second Eigen vector.

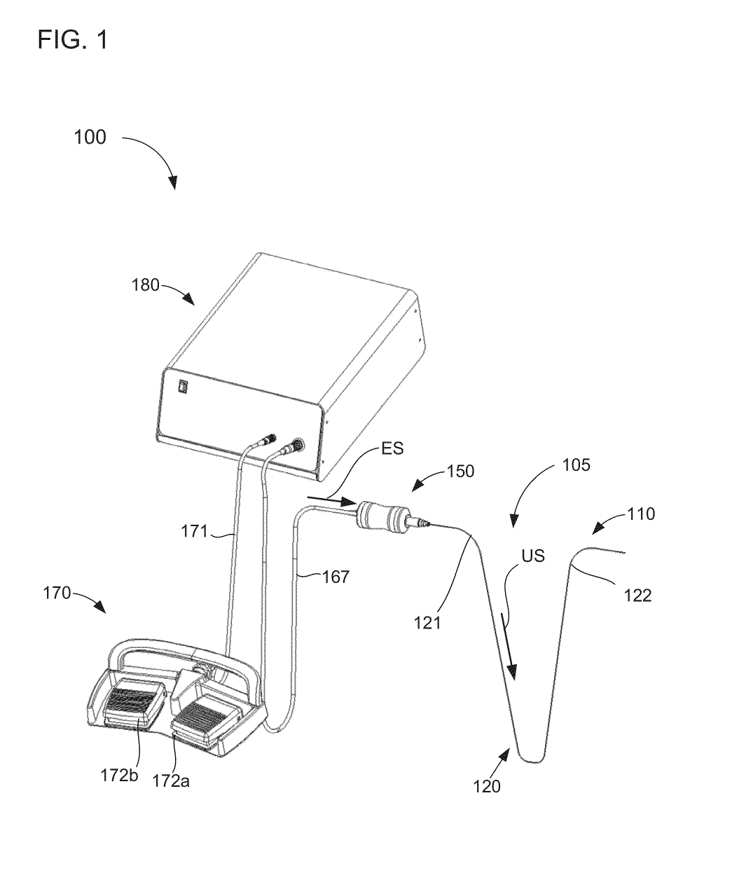

Embodiments described herein relate to ultrasonic energy ablation systems. In such systems a transmission member can be operably coupled to an ultrasonic energy source to deliver ultrasonic energy to a target bodily tissue. For example, FIG. 1 is an illustration of an ultrasonic energy ablation system 100, according to an embodiment. The ultrasonic energy ablation system 100 (also referred to herein as “ultrasonic system” or simply “system”) includes an ultrasonic generator 180, a foot switch 170, an ultrasonic transducer assembly 150, and a probe assembly 110. The ultrasonic generator 180 (or “generator”) can be any suitable generator configured to generate, control, amplify, and/or transfer an electric signal (e.g., a voltage) to the transducer assembly 150.

The ultrasonic generator 180 includes at least a processor, a memory and the circuitry (not shown in FIG. 1) to produce an electronic signal (i.e., a current and a voltage) having the desired characteristics that can be received by the ultrasonic transducer assembly 150 and converted into ultrasonic energy. In some embodiments, the ultrasonic generator 180 can be electrically coupled to (e.g., “plugged into”) an electric receptacle such that the ultrasonic generator 180 receives a flow of electric current. For example, in some embodiments, the ultrasonic generator 180 can be plugged into a wall outlet that delivers alternating current (AC) electrical power at a given voltage (e.g., 120V, 230V, or other suitable voltage) and a given frequency (e.g., 60 Hz, 50 Hz, or other suitable frequency).

Although not shown in FIG. 1, the ultrasonic generator 180 includes the electronic circuitry, hardware, firmware and or instructions to cause the ultrasonic generator 180 to act as a frequency inverter and/or voltage booster. In this manner, the ultrasonic generator 180 can produce and/or output a voltage to the transducer assembly 150 having the desired characteristics to produce the desired ultrasonic energy output. For example, in some embodiments, the ultrasonic generator 180 can receive AC electrical power at a frequency of approximately 60 Hz and a voltage of approximately 120V and convert the voltage to a frequency of up to approximately 20 kHz to 35 kHz with a voltage of approximately 500-1500 VAC (RMS). Thus, the ultrasonic generator 180 can supply the transducer assembly 150 with a flow of AC electrical power having an ultrasonic frequency.

As shown in FIG. 1, the system 100 includes the foot switch 170 that is in electric communication with the ultrasonic generator 180 via a foot switch cable 171. The foot switch 170 includes a first pedal 172a and second pedal 172b (collectively ‘172’) that are operative in controlling the delivery of the ultrasonic electrical energy supplied to the ultrasonic transducer assembly 150. For example, in some embodiments, a user (e.g., a physician, technician, etc.) can engage and/or depress one or more of the pedals 172 to control the current supplied to the ultrasonic transducer assembly 150 such that, in turn, the probe assembly 110 delivers the desired ultrasonic energy to the bodily tissue, as further described in detail herein.

In some embodiments, each of the first pedal 172a and the second pedal 172b can be configured to actuate different algorithms and/or pattern of ultrasonic electrical energy communicated to the ultrasonic transducer assembly 150. For example, the first pedal 172a can be operative to actuate an algorithm and/or a pattern of ultrasonic electrical energy configured to ablate soft stones (e.g., communicate a high pulse frequency and/or low amplitude ultrasonic vibrational energy). Similarly, the second pedal 172b can be operative to actuate an algorithm and/or a pattern of ultrasonic electrical energy configured to ablate hard stones (e.g., communicate a low pulse frequency and/or high amplitude ultrasonic electrical energy). In this manner, stones of different hardness can be ablated using the ultrasonic energy ablation system 100 without changing any component included in the ultrasonic generator 180 and/or the system 100. In some embodiments, as described herein, the ultrasonic energy ablation system 100 can include multiple probe assemblies 110 for use in different situations.

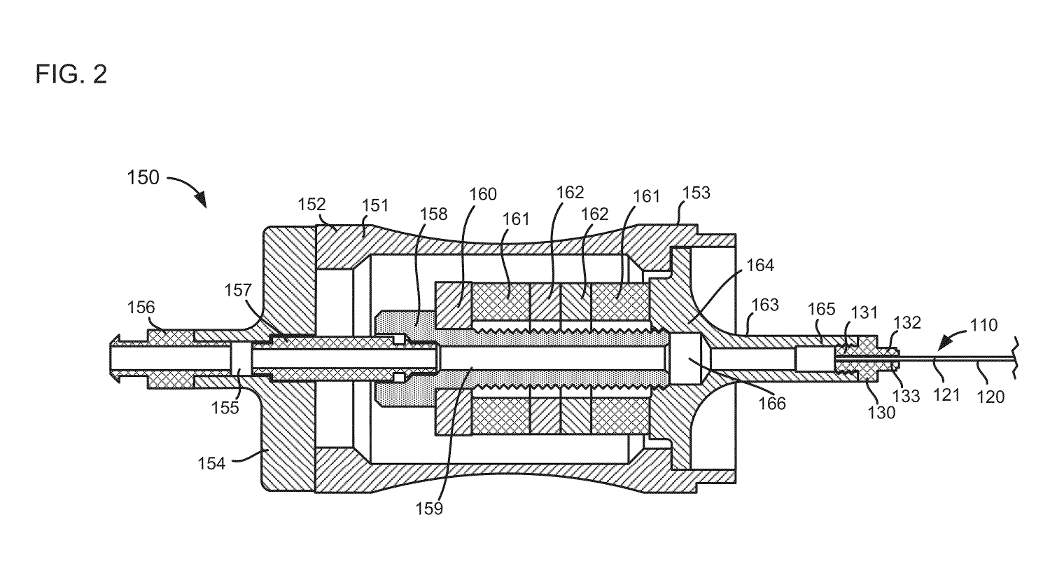

The transducer assembly 150 is in electric communication with the ultrasonic generator 180 via a transducer cable 167. In this manner, the transducer assembly 150 can receive an electrical signal (i.e., voltage and current) from the ultrasonic generator 180. The transducer assembly 150 is configured to produce and amplify the desired ultrasonic energy via a set of piezoelectric members 162 (i.e., piezoelectric rings) and an ultrasonic horn 163 (see e.g., FIG. 2), and transfer the ultrasonic energy to the probe assembly 110 and/or the transmission member 120. The transducer assembly 150 can be any suitable assembly of the types shown and described herein. In some embodiments, the transducer assembly can be operative to generate a vibrational frequency slightly above 20 kHz in conjunction with the probe assembly 110, for example, between 20 kHz and 21 kHz. Similarly stated, in some embodiments, the transducer assembly can be characterized by a natural frequency of between 20 kHz and 21 kHz. In some embodiments, the ultrasonic transducer assembly 150 and/or the ultrasonic generator 180 can include a control module configured to detect a resonant frequency of the probe assembly 110 and/or a transmission member 120 included in the probe assembly 110, coupled thereto. For example, a first transmission member can have a first flexural stiffness (e.g., is flexible) and is coupled to a first coupler to form a first probe assembly having a first resonant frequency (e.g., about 20.8 kHz). Similarly, a second transmission member can have a second flexural stiffness (e.g., is rigid) and is coupled to a second coupler to form a second probe assembly having a second resonant frequency (e.g., about 20.1 kHz). In such embodiments, the control module can be configured to detect the first resonant frequency and the second resonant frequency and produce a signal associated with (a) the first transmission member when the first probe assembly and thereby, the first transmission is coupled to the transducer assembly 150, and (b) the second transmission member when the second probe assembly and thereby, the second transmission member is coupled to the transducer assembly 150.

For example, in some embodiments, as shown in FIG. 2, the transducer assembly 150 includes a housing 151 having a proximal end portion 152 and a distal end portion 153. The housing 151 is configured to house or otherwise enclose at least a portion of a flow tube 157, a bolt 158, a back plate 160, a set of insulators 161, a set of piezoelectric rings 162, and a transducer horn 163.

The proximal end portion 152 of the housing 151 is coupled to a proximal cover 154 (e.g., via an adhesive, a press or friction fit, a threaded coupling, a mechanical fastener, or the like). The proximal cover 154 defines an opening 155 such that the proximal cover 154 can receive a portion of a connector 156 (e.g., a luer connector) on a proximal side thereof (e.g., substantially outside the housing 151) and a portion of the flow tube 157 on a distal side thereof (e.g., substantially inside the housing 151). Expanding further, the proximal cover 154 can receive the connector 156 and the flow tube 157 such that the proximal cover 154 forms a substantially fluid tight seal with the connector 156 and the flow tube 157. In this manner, a positive pressure and/or vacuum can be applied via the connector 156 to irrigate and/or aspirate the region of the body within which the probe assembly 110 is disposed. Similarly stated, this arrangement results in the connector 156 being placed in fluid communication with the lumen 122 defined by the transmission member 120.

The distal end portion 153 of the housing 151 is configured to receive the transducer horn 163 such that the transducer horn 163 is coupled to an inner surface of the housing 151. More specifically, the transducer horn 163 can be disposed at least partially within the housing 151 such that the transducer horn 163 can be moved relative to the housing 151 (e.g., when amplifying the ultrasonic energy), but not moved out of the housing 151 during normal use. The transducer horn 163 includes a proximal end portion 164 and a distal end portion 165 and defines a lumen 166 therethrough. The lumen 166 is configured to receive a portion of the bolt 158 at the proximal end portion 164 of the transducer horn 163 and a portion of the probe assembly 120 at the distal end portion 165 of the transducer horn 163, both of which are described in further detail herein.

As shown in FIG. 2, the back plate 160, the insulators 161, and the piezoelectric rings 162 are disposed within the housing 151 and about the bolt 158. More specifically, the arrangement of the back plate 160, the insulators 161, and the piezoelectric rings 162 is such that the back plate 160 is disposed proximal to the insulators 161 and the piezoelectric rings 162. The piezoelectric rings 162 are each disposed between the insulators 161. Similarly stated, a first insulator 161 is disposed proximal to the piezoelectric rings 162 and a second insulator 161 is disposed distal to the piezoelectric rings 162. The piezoelectric rings 162 are in electric communication (e.g., via wires not shown in FIGS. 1 and 2) with the ultrasonic generator 180, as described in further detail herein.

As shown in FIG. 2, a portion of the bolt 158 is configured to be disposed within the lumen 166 defined by the transducer horn 163. More specifically, the portion of the bolt 158 forms a threaded fit with an inner surface of the transducer horn 163 that defines the lumen 166. In this manner, the bolt 158 can be advanced within the lumen 166 such that the bolt 158 exerts a compressive force on the backing plate 160, the insulators 161, and the piezoelectric rings 162. Thus, the backing plate 160, the insulators 161, and the piezoelectric rings 162 are retained between a head of the bolt 158 (e.g., at the proximal end) and a proximal surface of the transducer horn 163. The torque applied to the bolt and/or the clamping force exerted between the head of the bolt 158 and the proximal surface of the transducer horn 163 is such that the deviation of the transducer natural frequency deviation is within ten percent from nominal. Therefore, in use, the piezoelectric rings 162 can vibrate and/or move the transducer horn 163, as further described herein.

The bolt 158 further defines a lumen 159 such that a proximal end portion of the bolt 158 can receive a distal end portion of the flow tube 157. In this manner, the lumen 159 defined by the bolt 158 and the flow tube 157 collectively place the lumen 166 defined by the transducer horn 163 in fluid communication with the connector 156. Thus, the lumen 166 of the transducer horn 163 can be placed in fluid communication with a volume substantially outside of the proximal end of the housing 151.

As shown in FIGS. 1 and 2, the probe assembly 110 includes at least a transmission member 120 and a coupler 130. The coupler 130 includes a first portion 131 and a second portion 132. The coupler defines a passageway 133 configured to fixedly receive a proximal end portion 121 of the transmission member 120. The first portion 131 is configured to be coupled to the transducer assembly 150. As shown in FIG. 2, the first portion 131 of the coupler 130 is disposed within the lumen 166 at the distal end portion 165 of the transducer horn 163 and forms a threaded fit with the inner surface of the transducer horn 163 that defines the lumen 166. In this manner, the probe assembly 110 can be removably coupled to the transducer assembly 150 via the coupler. The coupler 130 is configured to transfer at least a portion of an ultrasonic vibration produced by the transducer assembly 150 to the transmission member 120. The first portion 131 and the second portion 132 of the coupler 130 can be collectively configured to adjust a resonant frequency of the transmission member 120 and/or the probe assembly 110 (i.e., the transmission member 120 and the coupler 130 coupled thereto) to correspond to a vibrational frequency of the ultrasonic vibration produced by the transducer assembly 150. In some embodiments, the first portion 131 and the second portion 132 can be collectively configured to transform at least a portion of a linear component of the ultrasonic vibration provided by the transducer assembly 150 into a torsional component within the transmission member 120.

The transmission member 120 is an elongate tube having a proximal end portion 121 configured to be coupled with the coupler 130, and a distal end portion 122 (FIG. 1). A distal end of the distal end portion 122 of the transmission member is configured to contact or otherwise be placed proximate to a target bodily tissue and deliver ultrasonic vibrations to the target tissue. The transmission member 120 can be any suitable shape, size, or configuration and is described in further detail herein with respect to specific embodiments. In some embodiments, the transmission member 120 can optionally include any suitable feature configured to increase the flexibility (e.g., decrease the stiffness) of at least a portion of the transmission member 120, thereby facilitating the passage of the transmission member 120 through a tortuous lumen within a patient (e.g., a urinary tract, a vein, artery, etc.). For example, in some embodiments, a portion of the transmission member 120 can be formed from a material of lower stiffness than a different portion of the transmission member 120 formed from a material of greater stiffness. In some embodiments, the stiffness of at least a portion of the transmission member 120 can be reduced by defining an opening (e.g., a notch, a groove, a channel, a cutout, or the like), thereby reducing the area moment of inertia of the portion of the transmission member 120, as described herein with respect to specific embodiments. In some embodiments, the flexibility can be adjusted by varying a cross section (e.g., diameter) of at least a portion of the transmission member 120, for example, the outer cross section. The transmission member 120 can include any of the transmission members shown and described in U.S. Patent Publication No. 2014/0107534, entitled “Apparatus and Methods for Transferring Ultrasonic Energy to a Bodily Tissue,” filed on Oct. 16, 2012, which is incorporated by reference herein in its entirety.

In some embodiments, the coupler member 130 can have different shapes and sizes configured to match the vibrational frequencies of various transmission members 120 with the vibrational frequency of a vibration mode of the transducer assembly 150. This can allow transmission of a vibration mode and vibration frequency in a tight range to the bodily tissue, for example, in the range of between about 20 kHz and about 21 kHz, for example, about 20.1 kHz, 20.2 kHz, 20.3 kHz, 20.4 kHz, 20.5 kHz, 20.6 kHz, 20.7 kHz, 20.8 kHz, or about 20.9 kHz, inclusive of all ranges and values therebetween. In some embodiments, the coupler member 130 can also include one or more features on an outer surface of the coupler 130, for example, to match and/or adjust the natural frequencies of the probe assembly and/or transform at least a portion of a linear component of the ultrasonic vibration produced by the ultrasonic generator 180 into a torsional component within the transmission member 120. Such a torsional force can facilitate a function of the transmission member, for example, drilling through a bodily tissue, for example, a tissue within the vasculature of a subject, such as, for example, an occlusion, a clot, cancerous cells, tumors, aneurysms, plaque, fat deposits, lesions, thrombosis, calculi, uterine fibroids, bone metastases, adenomyosis, kidney stone, or any other bodily tissue and thereby disintegrating the bodily tissue.

In use, a user (e.g., a surgeon, a technician, physician, etc.) can operate the ultrasonic energy ablation system 100 to deliver ultrasonic energy to a target bodily tissue within a patient. The user can, for example, engage the pedals 172 of the foot switch 170 such that the ultrasonic generator 180 generates an alternating current (AC) and voltage with a desired ultrasonic frequency (e.g., 20,000 Hz). In this manner, the ultrasonic generator 180 can supply AC electric power to the piezoelectric rings 162. The AC electric power can urge the piezoelectric rings 162 to oscillate (e.g., expand, contract, or otherwise deform) at the desired frequency, which, in turn, causes the transducer horn 163 to move relative to the housing 151. Thus, with the probe assembly 110 coupled to the transducer horn 163, the movement of the transducer horn 163 vibrates and/or moves the probe assembly 110. In this manner, a distal end of the distal end portion 122 of the transmission member 120 can be disposed in contact with a portion of the patient adjacent to a target tissue such that the transmission member 120 transfers at least a portion of the ultrasonic energy to the target tissue (not shown in FIGS. 1 and 2). For example, in some embodiments, a distal tip of the transmission member 120 can impact a target tissue such as, for example, a kidney stone, a vascular occlusion, a blood clot, a portion of a bone, or the like, to break apart the occlusion. In some embodiments, the movement of the distal end portion 122 of the transmission member 120 is such that cavitations occur within the portion of the patient. In this manner, the cavitations can further break apart a target tissue. In some embodiments, the ultrasonic system 100 can optionally be used to aspirate and/or to supply irrigation to a target tissue site.

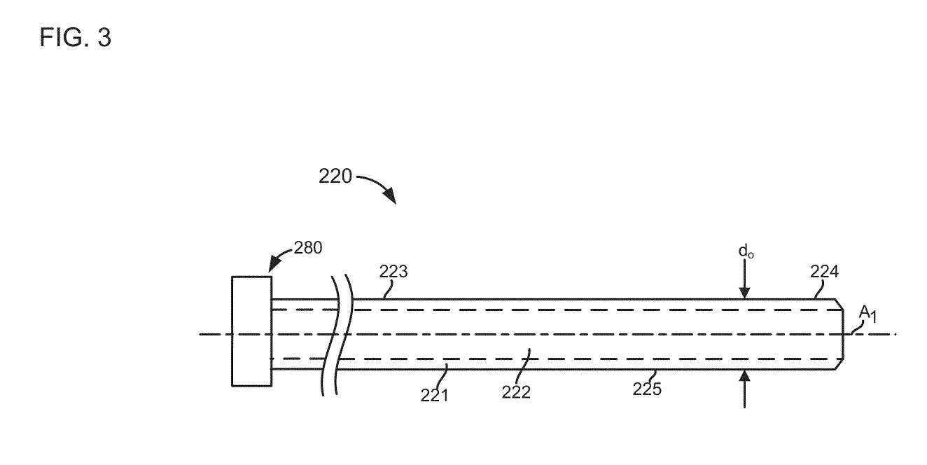

While described above in a general way, an ultrasonic energy system, such as the ultrasonic energy system 100, can include any suitable probe or transmission member of the types shown herein having increased flexibility to facilitate the passage of the transmission member through a tortuous lumen within a patient. For example, in some embodiments, a transmission member can have a suitable flexibility such that at least a portion of the transmission member can elastically (e.g., not permanently) deform within the tortuous anatomical structure. For example, FIG. 3 is a schematic illustration of a transmission member 220, according to an embodiment. The transmission member 220 can be included in any suitable ultrasonic energy system shown and described herein, such as, for example, the system 100 described above with reference to FIGS. 1 and 2. The transmission member 220 is a monolithically-constructed elongate member including a side wall 221 and defining a lumen 222 along a longitudinal axis A₁. In this manner, the transmission member 220 can provide aspiration from and/or irrigation (via the lumen 222, and the connecting lumens of any component to which the transmission member 220 is coupled) to a target tissue site during an ultrasonic procedure.

As shown in FIG. 3, the transmission member 220 includes a first portion 223, a second portion 224, and a third portion 225. The first portion 223 can be, for example, a proximal end portion, and can be at least operably coupled to an ultrasonic energy source 280, such as for example, the ultrasonic generator 180 and/or the transducer assembly 150 described above. For example, in some embodiments, the first portion 223 can be fixedly disposed within a passageway of a coupler (e.g., the coupler 130), as described above with reference to FIG. 2. In such embodiments, the coupler can be coupled to the ultrasonic energy source 280, thus, operably coupling the transmission member 220 to the ultrasonic energy source 280. The second portion 224 can be, for example, a distal end portion of the transmission member 220, and can be disposed within a body (not shown) to transfer ultrasonic energy from the first portion 223 into a bodily tissue.

The third portion 225 is disposed between the first portion 223 and the second portion 224. The third portion 225 can define a cross-sectional area moment of inertia that is less than a cross-sectional area moment of inertia of the first portion 223 and/or the second portion 224. In this manner, the transmission member 220 has a suitable flexural stiffness to be disposed along and/or within a tortuous path within the body such that the transmission member 220 efficiently and reliably transmits ultrasonic energy from the first portion 223 to the second portion 224. More particularly, the lower area moment of inertia of third portion 225 allows the third portion 225 to elastically deform more easily than the first portion 223 and/or the second portion 224. Said another way, the third portion 225 can bend (e.g., elastically) more easily about an axis that is perpendicular to the longitudinal axis A₁ of the transmission member 220 than can the first portion 223 and/or the second portion 224.

Moreover, the greater flexural stiffness of the first portion 223 and/or the second portion 224 can reduce losses of ultrasonic energy transmitted through the transmission member 220 that are associated with more flexible materials and/or members. Similarly stated, the spatial variation in the area moment of inertia results in higher transmission efficiency than would otherwise be obtained when forming the transmission member 220 to have a constant, lower flexural stiffness. Because the transmission member 220 is monolithically constructed, it is devoid of material interfaces that are known to cause reflection of the ultrasonic energy waves (and thereby inefficient transfer of the same). Additionally, because the transmission member 220 is monolithically constructed, there is a reduced likelihood that the transmission member 220 will fail during use as a result of discontinuities and/or stress concentration risers associated with the joining of separately constructed pieces.

The transmission member 220 can be formed from any suitable material such as, for example, Type 304 stainless steel, Type 316 stainless steel, nickel titanium alloy (nitinol), or any other super elastic metal or metal alloy. In some embodiments, the first portion 223, the second portion 224, and/or the third portion 225 can be formed from a material that is dissimilar from the material of the other portions. For example, in some embodiments, the first portion 223 and the second portion 224 can be formed from a first material and the third portion 225 can be formed from a second material. In such embodiments, the first material can have a modulus of elasticity that is substantially greater than the modulus of elasticity of the second material. For example, in some embodiments, the first portion 223 and the second portion 224 can be formed from Type 304 stainless steel and the third portion 225 can be formed from nitinol. In this manner, the first portion 223 and the second portion 224 can have a higher rigidity than that of the third portion 225. Similarly stated, the third portion 225 can have a lower flexural stiffness (defined above) than the flexural stiffness of the first portion 223 and the second portion 224.

In other embodiments, the monolithically-formed transmission member 220 can be formed from a substantially uniform material (e.g., a single material). Similarly stated, in some embodiments, the flexural stiffness of the first portion 223 and the second portion 224 can be greater than the flexural stiffness of the third portion 225 while being formed from the same material. In such embodiments, the spatial variation in the area moment of inertia is achieved by varying the cross-sectional size and/or shape of the transmission member 220 along its longitudinal axis A₁. For example, in some embodiments, the transmission member 220 can be substantially cylindrical and can have a uniform outer diameter dₒ along a length of the transmission member 220. Similarly stated, the first portion 223, the second portion 224, and the third portion 225 can each have substantially the same outer diameters dₒ. In such embodiments, the first portion 223, the second portion 224, and the third portion 225 can have a dissimilar inner diameter. For example, the first portion 223 and/or the second portion 224 can have an inner diameter that is smaller (resulting in a thicker sidewall 221) than the inner diameter of the third portion 225. Thus, the first portion 223 and/or the second portion 224 have an area moment of inertia that is greater than the area moment of inertia of the third portion 225. In this manner, the first portion 223 and/or the second portion 224 have a flexural stiffness that is greater than the flexural stiffness of the third portion 225.

In some embodiments, variations in the area moment of inertia can be achieved by varying the outer diameter dₒ while keeping a cross section of the lumen 222 defined by the transmission member constant. This can, for example, be used to define the flexural stiffness of the transmission member 210. For example a rigid transmission member 220 can be configured to define an outer diameter or otherwise cross-section dₒ₁ (e.g., about 0.18 inch) such that the rigid transmission member 220 has a high area moment of inertia and thereby, a high flexural stiffness. A flexible transmission member 220 can be configured to define an outer diameter or otherwise cross-section dₒ₂ such that dₒ₂<dₒ₁ (e.g., about 0.032 inch) and the flexible transmission member 220 has a low area moment of inertia and thereby, a low flexural stiffness. Similarly, a semi-flexible transmission member 220 can be configured to define an outer diameter of otherwise cross-section dₒ₃ such that dₒ₂<dₒ₃<dₒ₁ (e.g., about 0.063 inch) and the semi-flexible transmission member 220 has an intermediate area moment of inertia and thereby, an intermediate flexural stiffness relative to the rigid transmission member 220 and the flexible transmission member 220.

In other embodiments, the outer diameter of the first portion 223 and/or the outer diameter of the second portion 224 can be greater than the outer diameter of the third portion 225. Thus, by maintaining a similar inner diameter, the first portion 223 and/or the second portion 224 can have a greater area moment of inertia than the area moment of inertia of the third portion 225. In this manner, the first portion 223 and/or the second portion 224 have a flexural stiffness greater than the flexural stiffness of the third portion 225.

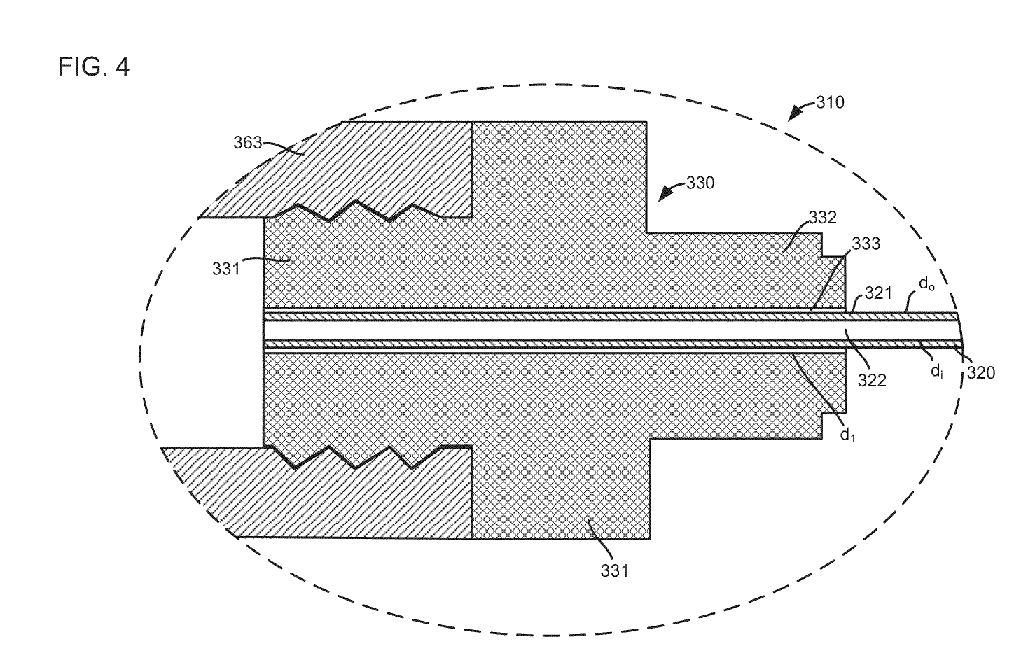

The proximal end portion of any of the transmission members described herein can be coupled to the coupler member (e.g., the coupler member 130) using any suitable mechanism. For example, as shown in FIG. 4, a probe assembly 310 can include at least a transmission member 320 and a coupler 330. The transmission member 320 can be substantially similar to the transmission member 120 described above with reference to FIGS. 1 and 2, thus, some portions of the transmission member 320 are not described in further detail herein.

The coupler 330 includes a first portion and a second portion 332 and defines a passageway 333 configured to fixedly receive a proximal end portion 321 of the transmission member 320. The first portion 331 is configured to be coupled to an ultrasonic energy source, for example, the transducer assembly 150 described with respect to the system 100. For example, as shown in FIG. 4, the first portion 331 can be configured to form a threaded coupling with a transducer horn, for example, the transducer horn 363, as described above in detail with reference to FIG. 2. The passageway 333 has a diameter that can be any suitable size. In this manner, the coupler 330 can be configured to receive (within the passageway 333) the proximal end portion 321 of the transmission member 320, as described in further detail herein. The coupler 330 is configured to transfer at least a portion of an ultrasonic vibration produced by the ultrasonic energy source to the transmission member 320. Similarly stated, the coupler 330 defines a path through which ultrasonic energy can be conveyed from the ultrasonic energy source to the transmission member 320. Furthermore, the first portion 331 and the second portion 332 are configured to adjust a resonant frequency of the transmission member 330 and or the probe assembly 310, to correspond to a vibrational frequency of the ultrasonic vibration produced by the ultrasonic energy source. Said another way, the first portion 331 and the second 332 portion can be shaped and sized to adjust the resonant frequency of the probe assembly 310, or the transmission member 320 to correspond to a vibrational frequency of the ultrasonic vibration produced by the ultrasonic energy source.

In some embodiments, the outer surface of the first portion 331 can be discontinuous from the outer surface of the second portion 332. For example, in some embodiments, the first portion 331 can have first diameter and a first length, and the second portion 332 can have a second diameter and a second length. The first diameter can be greater than the second diameter. Furthermore, a ratio of the first length to the second length can be such that the resonant frequency of the transmission member 320 or otherwise the probe assembly 310 can be in the range of about 20 kHz to about 21 kHz, for example, about 20.1 kHz, 20.2 kHz, 20.3 kHz, 20.4 kHz, 20.5 kHz, 20.6 kHz, 20.7 kHz, 20.8 kHz, or about 20.9 kHz, inclusive of all ranges and values therebetween. In some embodiments, the transmission member 320 can be a semi-flexible transmission member and the coupler can be configured to adjust the resonant frequency of the semi-flexible transmission member or otherwise a probe assembly that includes the semi-flexible transmission member to be about 20.8 kHz. In other embodiments, the transmission member 320 can be a rigid transmission member and the coupler 330 can be configured to adjust the resonant frequency of the transmission member or otherwise that includes the rigid transmission member to be about 20.1 kHz.

While not shown, in some embodiments, the coupler 330 can include a third portion disposed between the first portion 331 and the second portion 332. The third portion can have a third diameter and a third length. The third diameter can be less than the first diameter and greater than the second diameter such that the coupler is discontinuous. In such embodiments, the ratio of the first length, the second length, and the third length can be such that the resonant frequency of the transmission member 330 or otherwise the probe assembly 310 is in the range of about 20 kHz to about 21 kHz. In some embodiments, a ratio of the first length and the second length can be in the range of about 2.0, 2.1, 2.2, 2.3, 2.4, 2.5, 2.6, 2.7, 2.8, 2.9, or about 3.0, inclusive of all ranges therebetween. For example, in some embodiments, the ratio of the first length to the second length can be about 2.35. Furthermore, a ratio of the first length and the third length can be about 0.55, 0.56, 0.57, 0.58, 0.59, 0.60, 0.61, 0.62, 0.63, 0.64, or about 0.65, inclusive of all ranges therebetween. For example, in some embodiments, the ratio of the first length and the third length can be about 0.61. In such embodiments, the transmission member 330 can be, for example, a rigid transmission member, and the coupler 330 can be configured to adjust the frequency of the rigid transmission member or otherwise probe assembly 310 to a first resonant frequency, for example, about 20.1 kHz.

In some embodiments, a ratio of the first length and the second length can be in the range of about 0.78, 0.79, 0.80, 0.81, 0.82, 0.83, 0.84, 0.85, 0.86, 0.87, or about 0.88, inclusive of all ranges therebetween For example, in some embodiments, the ratio of the first length to the second length can be about 0.83. Furthermore, a ratio of the first length and the third length can be about .0.92, 0.94, 0.96, 0.98, 1.0, 1.02, 1.04, 1.06, 1.08, or about 1.10 inclusive of all ranges therebetween. For example, in some embodiments, the ratio of the first length and the third length can be about 1. In such embodiments, the transmission member can be, for example, a semi-flexible transmission member and the coupler 330 can be configured to adjust the frequency of the semi-flexible transmission member or otherwise probe assembly 310 to a second resonant frequency, for example, about 20.8 kHz.

In some embodiments, at least a portion of the ultrasonic vibration produced by the ultrasonic energy source (e.g., the transducer assembly 150) can include a linear component (e.g., along a longitudinal axis of the transmission member 320). The first portion 331 and the second portion 332 of the coupler 330 can be collectively configured to transform at least a portion of the linear component of the ultrasonic vibration into a torsional component within the transmission member 330. For example, in some embodiments, the coupler 330 can include a third portion disposed between the first portion and the second portion. The third portion can define a groove such that the first portion, the second portion, and the third portion are collectively configured to transform at least a portion of the linear component of the ultrasonic vibration into a torsional component within the transmission member 330, for example, to produce a torsional vibratory force. In some embodiments, the groove can be a circumferential groove having a width and a depth configured to produce the torsional vibratory force. For example, in some embodiments, the width of the groove can be in the range of about 0.1 inches, 0.11 inches, 0.12 inches, 0.13 inches, 0.14 inches, 0.15 inches, 0.16 inches, 0.17 inches, 0.18 inches, 0.19 inches, or about 2.0 inches inclusive of all ranges therebetween. For example, in some embodiments, the width of the groove can be about 0.15 inches. In some embodiments, the groove can be a helical groove having an angular width and a cut angle, configured to produce the torsional vibratory force. The helical groove can be a straight cut helical groove or a curved cut helical groove.

In some embodiments, the coupler 330 can also define a lateral opening on a sidewall of the coupler 330, for example, on a sidewall of a third portion of the coupler 330. The lateral opening can be in fluidic communication with the passageway 333 and positioned to be in fluidic communication with a lumen 322 defined by the transmission member 320. For example, the transmission member 320 can also include a lateral opening on a sidewall of the proximal end portion 321 of the transmission member 320. The proximal end portion 321 of the transmission member 320 can be disposed in the passageway 333 defined by the coupler 330 and positioned such that the lateral opening defined in the proximal end portion 321 is substantially adjacent to the lateral opening of the coupler 330. In this manner, the lateral opening of the coupler 330 can be in fluidic communication with the lumen 322 of the transmission member 320.

The transmission member 320 includes a proximal end portion 321 and a distal end portion (not shown in FIG. 4), and defines a lumen 322 therethrough. The transmission member 320 can be any suitable shape, size, or configuration. For example, in some embodiments, at least a portion of the transmission member 320 is substantially annular and includes an outer diameter dₒ and an inner diameter dᵢ. In some embodiments, the size and shape of the transmission member 320 (e.g., the outer diameter dₒ) can substantially correspond to the size and shape (e.g., the diameter d₁) of the lumen 333 defined by the coupler 330 such that the proximal end portion 321 of the transmission member 320 can be disposed therein.

For example, in some embodiments, the diameter d₁ of the lumen 333 can be greater than the outer diameter dₒ of the transmission member 320, thus, the transmission member 320 can be disposed within the lumen 333 of the coupler 330. Furthermore, with the diameter d₁ of the lumen 333 greater than the outer diameter dₒ of the transmission member 320 an adhesive can be disposed within a void between the transmission member 320 and the inner surface of the coupler 330. Thus, the transmission member 320 can be fixedly coupled to the coupler 330 without the need for crimping, applying a compressive force to the transmission member or the like. Expanding further, the transmission member 320 can be fixedly coupled to the coupler 330 without plastically (e.g., permanently) deforming the transmission member 320, thereby decreasing the likelihood of failure and also decreasing losses due to reflections of ultrasonic energy produced by discontinuity. In other embodiments, the transmission member 320 can be coupled via welding or brazing while still realizing the benefits described herein.

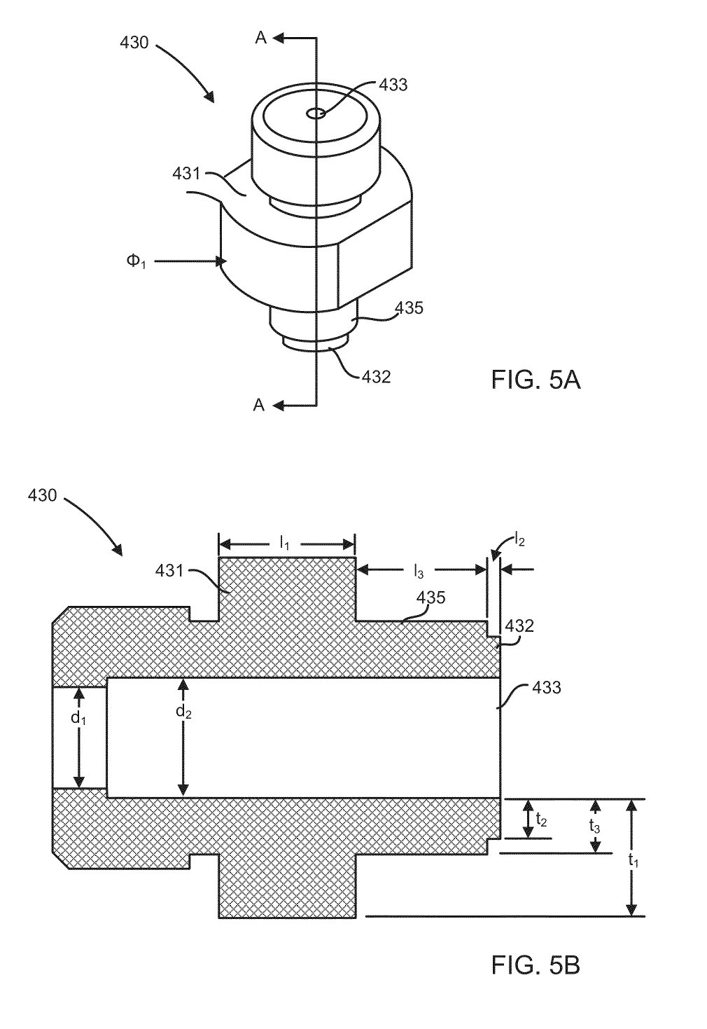

In some embodiments, a coupler can define a size and/or a shape to adjust the vibrational mode and natural frequency of a transmission member assembly. Similarly stated, the coupler can compensate for a transmission member having a predetermined area moment of inertia, flexibility, mass, flexural stiffness, length or the like, which can result in the transmission member having a natural frequency outside of a desired range, by adjusting the natural frequency of the transmission member assembly to match the frequency performance of a transducer assembly. For example, FIG. 5A shows a perspective view of a coupler 430 configured to couple a first transmission member having a first flexural stiffness, for example a flexible transmission member having an outer diameter of about 0.032 inch as described before herein, to a transducer assembly, for example, the transducer assembly 150 shown with respect to FIG. 2. FIG. 5B shows a cross sectional view of the coupler 430 taken along line A-A. The coupler 430 includes a first portion 431, a second 432, and a third portion 435. The first portion 431 is configured to be coupled to an ultrasonic energy source, for example, the transducer assembly 150 shown and described with respect to FIG. 2. For example, the first portion 431 can include a threaded portion configured to form a coupling with a transducer horn, for example, the transducer horn 163 included in the transducer assembly 150. The number of threads on the threaded portion can be, for example, between about three and four. An outer surface of the first portion 431 is discontinuous from an outer surface of the second portion 432, and an outer surface of the third portion 435. The first portion 431 defines a length l₁, for example, of about 0.125 inch. A first portion of an outside wall of the first portion 431 can be substantially flat and define a thickness t₁, for example, of about 0.113 inch. A second portion of the outside wall of the first portion 431 can be substantially arcuate and can define an arc radius of about 0.15 inch. The second portion 432 can define a length l₂, for example, of about 0.025 inch and a thickness t₂, for example, of about 0.034 inch. The third portion 435 can define a length l₃, for example, of about 0.123 inch and a thickness t₃, for example, of about 0.051 inch. A first portion of the lumen 433 within the threaded portion can define a first diameter d₁, for example, of about 0.025 inch. A remaining second portion of the lumen 433 can define a second diameter d₂, for example, of about 0.037 inch.

The coupler 430 defines a passageway 433 configured to fixedly receive a proximal end portion of a first transmission member, for example, a flexible transmission member, using any suitable coupling method as described herein. Furthermore, the passageway 433 can be in fluidic communication with an irrigation lumen of the first transmission member. The coupler 430 is configured to transfer at least a portion of an ultrasonic vibration produced by an ultrasonic energy source to the first transmission member. The first portion 431, the second portion 432, and the third portion 435 are collectively configured to adjust a resonance frequency of the first transmission member, or a first probe assembly (i.e., the coupler 430 with the first transmission member coupled thereto), to correspond to a vibrational frequency of the ultrasonic vibration produced by the ultrasonic energy source (e.g., the transducer assembly 150). In some embodiments, the coupler 430 is operative to adjust the resonant frequency of the first transmission member to correspond to the ultrasonic energy source, such that the transmitted vibrational frequency is between 20 kHz and 21 kHz (e.g., about 20.9 kHz). Similarly stated, the coupler 430 is configured to adjust a resonant frequency of the first transmission member or otherwise the first probe assembly to be in the range of about 20 kHz and 21 kHz.

In some embodiments, the lengths of the first portion 431, the second portion 432, and the third portion 433 can be adjusted to adjust the resonant frequencies of transmission members of otherwise probe assemblies to a desired value or range. For example, a ratios of the length of the first portion 431, to the length of the second portion 432, and/or the length of the third portion 435 can be such that the resonant frequency of a transmission member or otherwise the probe assembly can be adjusted to a desired value.

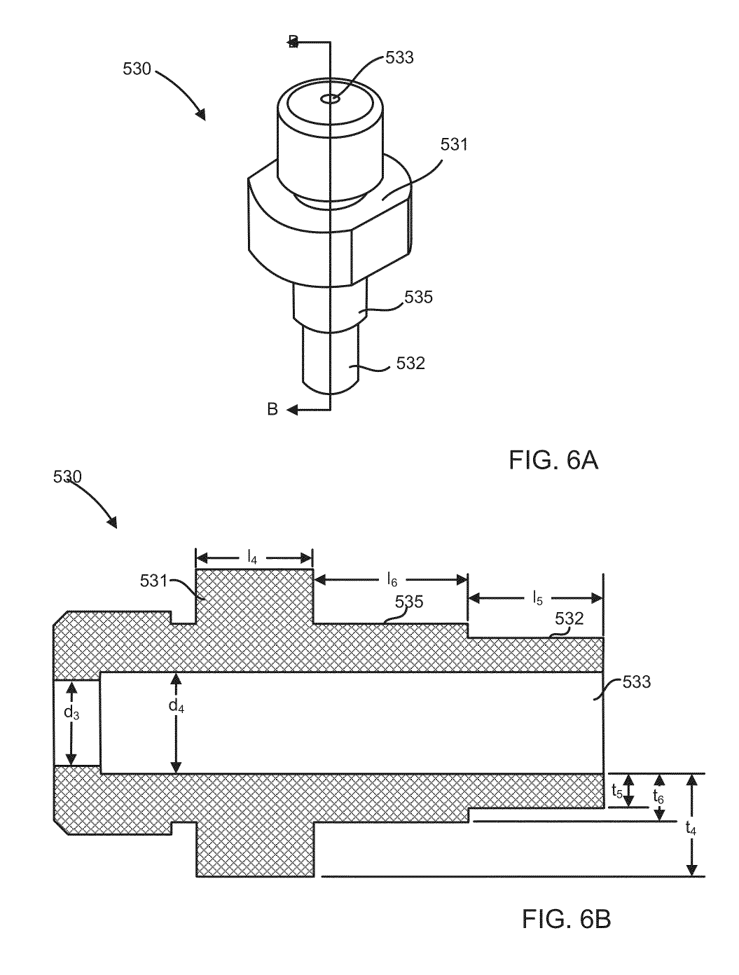

For example, FIG. 6A shows a perspective view of a coupler 530 and FIG. 6B shows a cross sectional view of the coupler 530 taken along line B-B. The coupler 530 includes a first portion 531, a second portion 532, and a third portion 535. The coupler 530 defines a passageway 533 configured to receive a proximal end portion of a transmission member. The transmission member can be a second transmission member having a second flexural stiffness (e.g., a semi-flexible transmission member), or a third transmission member having a third flexural stiffness (e.g., a rigid transmission member). The passageway 533 can also be in fluidic communication with an irrigation lumen of the transmission member, for example, to allow irrigation and/or aspiration of a target tissue. A first portion of the passageway 533 within the threaded portion can define a first diameter d₃, for example, of about 0.025 inch. A remaining second portion of the passageway 533 can define a second diameter d₄, for example, of about 0.034 inch. The coupler 530 is configured to be coupled to an ultrasonic energy source, for example, the transducer assembly 150 shown and described with respect to FIG. 2. For example, the first portion 531 can include a threaded portion configured to form a coupling with a transducer horn, for example transducer horn 163, as described above in detail with reference to FIG. 2. The number of threads on the threaded portion can be, for example, between three and four. The coupler 530 is configured to transfer at least a portion of an ultrasonic vibration produced by the ultrasonic energy source to the transmission member. The first portion 531, the second portion 532, and the third portion 535 can be collectively configured to adjust a resonant frequency of the transmission member or otherwise a probe assembly (i.e., the coupler 530 with the transmission member coupled thereto) to correspond to a vibrational frequency of the ultrasonic vibration produced by the ultrasonic energy source. For example, the first portion 531, the second portion 532, and the third portion 535 can be collectively configured to adjust the resonant frequency of the transmission member (e.g., the second transmission member or the third transmission member) or the probe assembly (e.g., a second probe assembly including the coupler 530 and the second transmission member, or a third probe assembly included the coupler 530 and the third transmission member) to be in the range of about 20 kHz to about 21 kHz.

The first portion 531 has a first diameter (or otherwise cross-section) and a first length l₄, the second portion 532 has a second diameter (or otherwise cross-section) and a second length l₅, and the third portion 535 has a third diameter (or otherwise cross-section) and a third length l₆. The second diameter is larger than the third diameter but smaller than the first diameter. A sidewall of the first portion 531 can have a first thickness t₄, a sidewall of the second portion 532 can have a second thickness t₅, and a sidewall of the third portion 535 can have a third thickness t₆, such that t₄>t₆>t₅. In some embodiments, the first thickness t₄ can be about 0.113 inches. In some embodiments, the second thickness can be about 0.034 inches. In some embodiments, the third thickness can be about 0.051 inches.

A ratio of the first length l₄ and the second length l₅, and/or the ratio between the first length l₄ and the third length l₆ can be varied to adjust the resonant frequency of the transmission member or otherwise probe assembly to a predetermined resonant frequency, for example, in the range of about 20 kHz to about 21 kHz. In some embodiments, a ratio of the first length l₄ and the second length l₅ can be about 2.0, 2.1, 2.2, 2.3, 2.4, 2.5, 2.6, 2.7, 2.8, 2.9, or about 3.0, inclusive of all ranges therebetween. For example, in some embodiments, the first length l₄ can be about 0.148 inches, and the second length l₅ can be about 0.063 inches such that the ratio of the first length l₄ to the second length l₅ is about 2.35. Furthermore, a ratio of the first length l₄ and the third length l₆ can be about 0.55, 0.56, 0.57, 0.58, 0.59, 0.60, 0.61, 0.62, 0.63, 0.64, or about 0.65, inclusive of all ranges therebetween. For example, in some embodiments, the first length l₄ can be about 0.148 inches and the third length l₆ can be about 0.188 inches such that the ratio of the first length l₄ and the third length l₆ is about 0.61. In such embodiments, the transmission member can be, for example, the third transmission member, i.e., the rigid transmission member, and the coupler 530 can be configured to adjust the frequency of the rigid transmission member or otherwise third probe assembly to a second third frequency, for example, about 20.1 kHz.

In some embodiments, a ratio of the first length l₄ and the second length l₅ can be about 0.78, 0.79, 0.80, 0.81, 0.82, 0.83, 0.84, 0.85, 0.86, 0.87, or about 0.88, inclusive of all ranges therebetween. For example, in some embodiments, the first length l₄ can be about 0.125 inches and the second length l₅ can also be about 0.150 inches such that the ratio of the first length l₄ to the second length l₅ is about 0.83. Furthermore, a ratio of the first length l₄ and the third length l₆ can be about 0.92, 0.94, 0.96, 0.98, 1.0, 1.02, 1.04, 1.06, 1.08, or about 1.10 inclusive of all ranges therebetween. For example, in some embodiments, the first length l₄ can be about 0.125 inches and the third length l₆ can be about 0.125 inches such that the ratio of the first length l₄ and the third length l₆ is about 1. In such embodiments, the transmission member can be, for example, the second transmission member i.e., the semi-flexible transmission member and the coupler 530 can be configured to adjust the frequency of the semi-flexible transmission member or otherwise probe assembly to a second resonant frequency, for example, about 20.8 kHz.

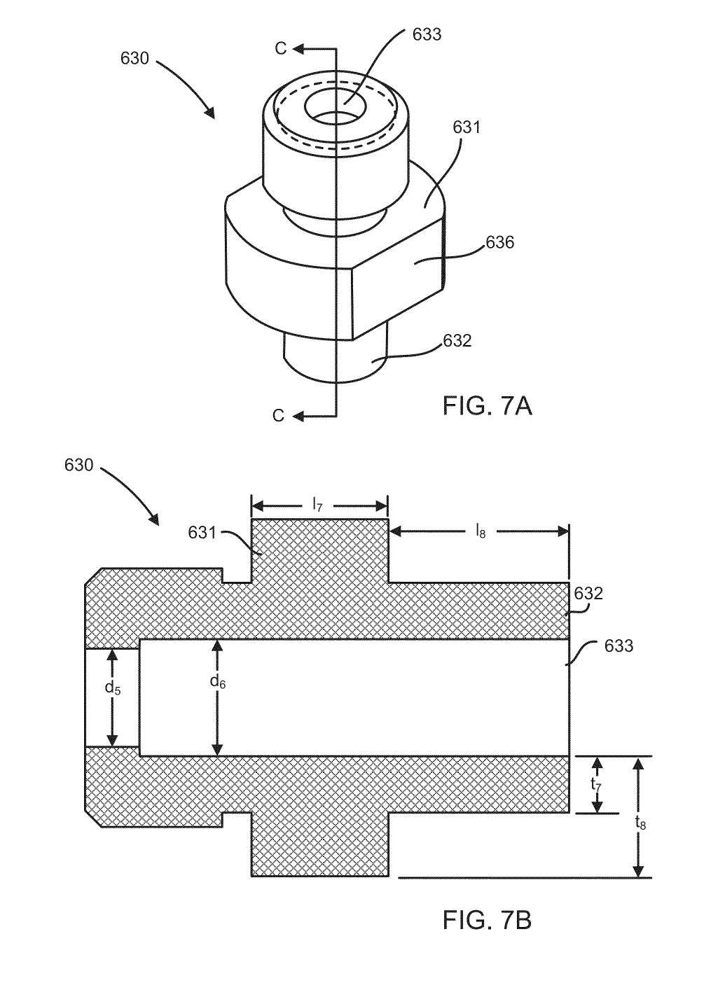

In some embodiments, a coupler can have a size and a shape to match the vibrational mode and vibrational frequency of a second (or “semi-flexible”) transmission member as described before herein to a transducer assembly, for example, transducer assembly 150. FIG. 7A shows a perspective view of a coupler 630 configured to couple a semi-flexible transmission member, for example a transmission member having an outer diameter of about 0.063 inch as described before herein, to a transducer assembly, for example, the transducer assembly 150 shown with respect to FIG. 2. FIG. 7B shows a cross sectional view of the coupler 630 taken along line C-C. The coupler 630 includes a first portion 631 and a second portion 632. The coupler 630 defines a passageway 633 configured to receive a proximal end portion of a transmission member, for example, the transmission member 120, 220, 320, or any other transmission member described herein. In some embodiments, the transmission member can be a semi-flexible transmission member. The first portion 631 is configured to be coupled to an ultrasonic energy source, for example, the transducer assembly 150, or any other transducer assembly described herein. For example, the first portion 631 can include a threaded portion configured to form a coupling with a transducer horn, for example, transducer horn 163, as described above in detail with reference to FIG. 2. The number of threads on the threaded portion can be between three and four. The coupler 630 is configured to transfer at least a portion of an ultrasonic vibration produced by the ultrasonic energy source to the transmission member. Furthermore, the first portion 631 and the second portion 632 are collectively configured to adjust a resonant frequency of the transmission member or otherwise probe assembly (i.e., the coupler 630 coupled to the transmission member) to correspond to a vibrational frequency of the ultrasonic vibration produced by the ultrasonic energy source. For example, in some embodiments, the first portion 631 and the second portion 632 can be collectively configured to adjust the resonant frequency of the transmission member or otherwise the probe assembly to be in the range of about 20 kHz to about 21 kHz.

A first portion of the passageway 633 can define a first diameter d₅, for example, of about 0.052 inch. A remaining second portion of the lumen 633 can define a second diameter d₆, for example, of about 0.066 inch. The first portion 631 can be define a first length l₇ and the second portion 632 can be define a second length l₈ such that a ratio between the first length l₇ and the second length l₈ is about 5. For example, the first length l₇ can be about 0.125 inch, and the second length l₈ can be about 0.025 inch. In such embodiments, the coupler 630 can be configured to be coupled to a semi-flexible transmission member and adjust the resonant frequency of the semi-flexible transmission or otherwise a probe assembly that includes the semi-flexible transmission member to be about 20.9 kHz. A portion of an outside wall of the first portion 631 can be substantially flat and define a thickness t₇ of about 0.100 inch. A second portion of the outside wall of the first portion 631 can be substantially arcuate and can define an arc radius of about 0.15 inch. The second portion 632 can define a thickness t₈, for example, of about 0.036 inch.

In some embodiments, the coupler 630 can be configured to have a size and shape to match the vibrational mode and vibrational frequency of a third (or “rigid”) transmission member as described before herein to a transducer assembly, for example, transducer assembly 150. In such embodiments, a first portion of the lumen 633 within the threaded portion can define a first diameter d₅ of about 0.106 inch. A remaining second portion of the lumen 633 can define a second diameter d₆ of about 0.125 inch. The proximal portion 631 defines a length l₇ of about 0.150 inch and a thickness t₇ of about 0.093 inch. The distal portion 632 can define a length l₈ of about 0.158 inch and a thickness t₈ of about 0.028 inch. The reconfigured coupler 630 can be fixedly coupled to the rigid transmission member using any suitable coupling method as described herein. In such configurations, the coupler 630 is operative to match the vibrational mode and vibrational frequency of the rigid transmission member to the transducer assembly, such that the resonant frequency of the rigid transmission member or a probe assembly that includes the rigid transmission member is about 20.9 kHz.

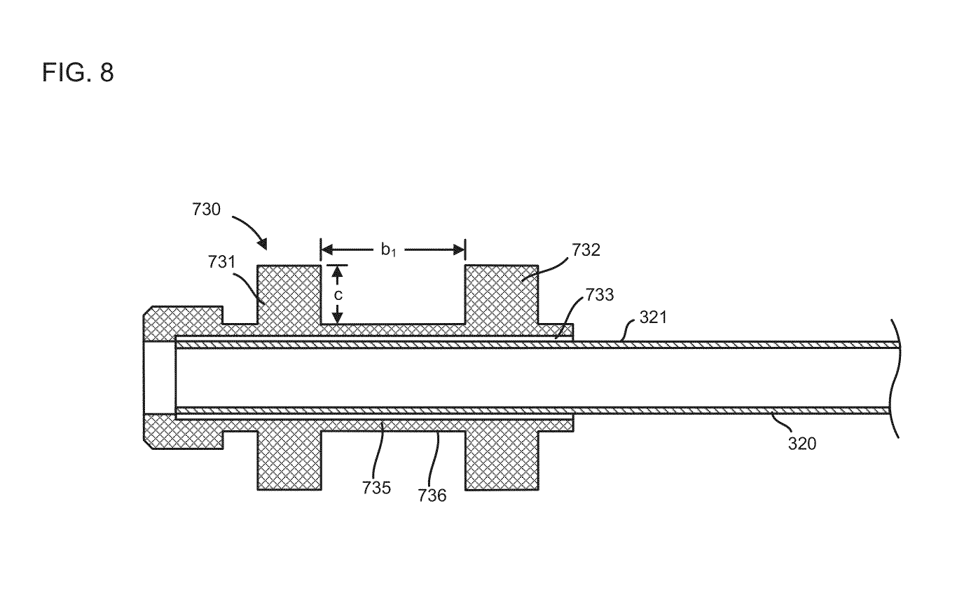

In some embodiments, a coupler can include features on a surface of the coupler, configured to transform at least a portion of a linear component of an ultrasonic vibration produced by an ultrasonic energy source into a torsional vibration within a transmission member. The torsional component of the vibration can produce a torsional force which can facilitate a function of the transmission member, for example, drilling through a tissue such as, for example, a clot in the vasculature. For example, FIG. 8 shows a cross section of a coupler 730. The coupler 730 includes a first portion 731, a second portion 732, and a third portion 735 disposed between the first portion 731 and the second portion 732. The coupler 730 defines a passageway 733 configured to fixedly receive a proximal end portion 321 of a transmission member 320, for example, a flexible transmission member, or any other transmission member described herein. The first portion 731 is configured to be coupled to an ultrasonic energy source, for example, the transducer assembly 150 shown and described with respect to FIG. 2. For example, the first portion 731 can included a threaded portion configured to form a coupling with a transducer horn, for example transducer horn 163, as described above in detail with reference to FIG. 2. The coupler 730 can be configured to transfer at least a portion of an ultrasonic vibration produced by the ultrasonic energy source to the transmission member 320. The ultrasonic vibration produced by the ultrasonic energy source can be a linear vibration, i.e., includes a linear component.

The third portion 735 defines a groove 736, which can, for example, be a circumferential groove. Thus, the coupler 730 defines a shape which resembles that of a “dumbbell.” In this manner, an outer surface of the first portion 731 can be discontinuous from the outer surface of the second portion 732 and the third portion 735. The groove 736 has a width b₁ and a depth c. In some embodiments, the width b₁ can be about 0.1 inches, 0.11 inches, 0.12 inches, 0.13 inches, 0.14 inches, 0.15 inches, 0.16 inches, 0.17 inches, 0.18 inches, 0.19 inches, or about 0.20 inches, inclusive of all ranges therebetween. For example, in some embodiments, the width b₁ can be about 0.15 inches. The groove 736 can be shaped and sized such that the first portion 731, the second portion 732, and the third portion 735 are collectively configured to transform at least a portion of the linear component of the ultrasonic vibration into a torsional component within the transmission member 320 (e.g., a flexible transmission member). Similarly stated, the coupler 730 can be configured to receive only the linear component of the ultrasonic vibration from the ultrasonic energy source and transform at least a portion of the linear component into a torsional component within the transmission member 320. Thus, the coupler 730 can, for example, induce a bimodal vibration in the transmission member 320 that has the torsional component coupled with the linear component. In some embodiments, substantially all of the linear component of the ultrasonic vibration can be transformed into the torsional component such that the ultrasonic vibration produced by the transmission member 320 is substantially the torsional component of the ultrasonic vibration. The torsional component of the vibration, or the bimodal vibration described herein can be particularly effective for intravascular ultrasonic ablation therapies, for example, breaking vascular clots, cancerous cells, adipose tissue, etc.

Furthermore, the width b₁ and the depth c can be varied such that the first portion 731, the second portion 732, and the third portion 735 are collectively configured to adjust a resonant frequency of the transmission member 320, or otherwise a probe assembly that includes the transmission member 320 to correspond to a vibrational frequency of the ultrasonic vibration produced by the ultrasonic energy source. For example, the first portion 731, the second portion 732, and the third portion 735 can be collectively configured to adjust the resonant frequency of the transmission member 320, or otherwise a probe assembly that includes the transmission member 320 to be in the range of about 20 kHz to about 21 kHz.

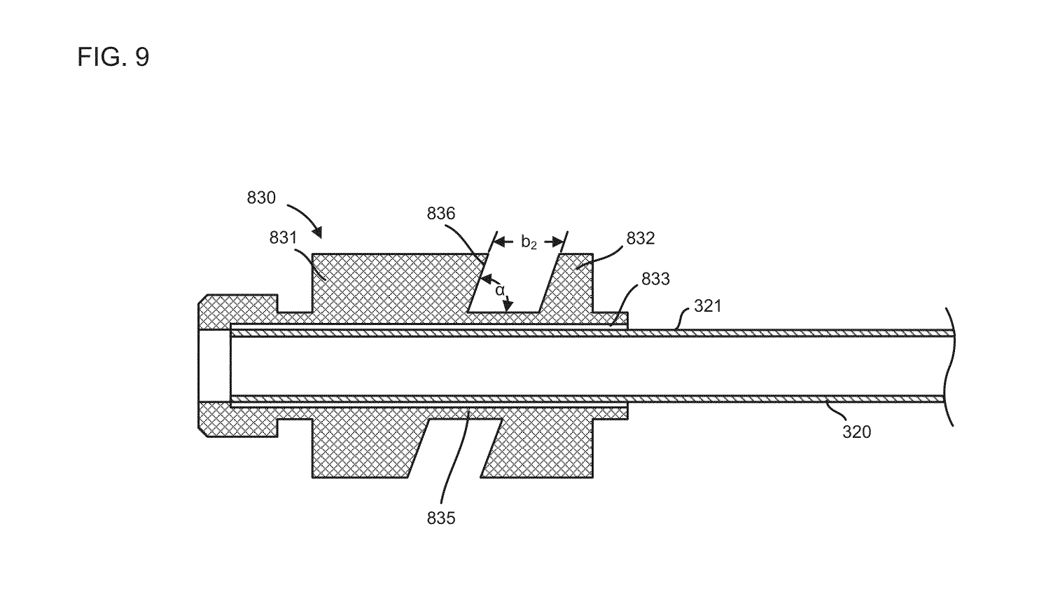

In some embodiments, a coupler can include one or more helical grooves on a surface of the coupler, configured to transform at least a portion of a linear component of an ultrasonic vibration produced by an ultrasonic energy source into a torsional vibration within a transmission member. For example, FIG. 9 shows a cross section of a coupler 830. The coupler 830 includes a first portion 831, a second portion 832, and a third portion 835 disposed between the first portion 831 and the second portion 832. The coupler 830 defines a passageway 833 configured to fixedly receive a proximal end portion 321 of a transmission member 320, for example, a flexible transmission member, or any other transmission member described herein. The first portion 831 is configured to be coupled to an ultrasonic energy source, for example, the transducer assembly 150 shown and described with respect to FIG. 2. For example, the first portion 831 can include a threaded portion configured to form a coupling with a transducer horn, for example transducer horn 163, as described above in detail with reference to FIG. 2. The coupler 830 can be configured to transfer at least a portion of an ultrasonic vibration produced by the ultrasonic energy source to the transmission member 320. The ultrasonic vibration produced by the ultrasonic energy source can be a linear vibration, i.e., includes a linear component.

The third portion 835 defines a straight cut helical groove 836. Thus, an outer surface of the first portion 831 can be discontinuous from the outer surface of the second portion 832 and the third portion 835. The groove 836 defines a width b₂ and a cut angle α. In some embodiments, the width b₂ can be in the range of about 0.04 inch to about 1.97 inch. In some embodiments, the cut angle α can be in the range of about 0 degrees to about 180 degrees. As shown in FIG. 9, the coupler 830 includes a single straight cut helical groove. In some embodiments, the coupler 830 can include a set of curved cut helical grooves, e.g., 2, 3, 4, 5, or even more. The groove 836 can sized such that the first portion 831, the second portion 832 and the third portion 835 are collectively configured to transform at least a portion of the linear component of the ultrasonic vibration into a torsional component within the transmission member 320 (e.g., a flexible transmission member). Similarly stated, the coupler 830 can be configured to receive only the linear component of the ultrasonic vibration from the ultrasonic energy source and transform at least a portion of the linear component into the torsional component within the transmission member 320. Thus, the coupler 830 can, for example, induce a bimodal vibration in the transmission member that has the torsional component coupled with the linear component. In some embodiments, substantially all of the linear component of the ultrasonic vibration can be transformed into the torsional component such that the ultrasonic vibration produced by the transmission member 320 is substantially the torsional component of the ultrasonic vibration. The torsional component of the vibration, or the bimodal vibration described herein can be particularly effective for intravascular ultrasonic ablation therapies, for example, breaking vascular clots, cancerous cells, adipose tissue, etc.

Furthermore, the width b₂ and the cut angle α can be varied such that the first portion 831, the second portion 832, and the third portion 835 are collectively configured to adjust a resonant frequency of the transmission member 320, or otherwise a probe assembly that includes the transmission member 320 to correspond to a vibrational frequency of the ultrasonic vibration produced by the ultrasonic energy source. For example, the first portion 831, the second portion 832, and the third portion 835 can be collectively configured to adjust the resonant frequency of the transmission member 320, or otherwise a probe assembly that includes the transmission member 320 to be in the range of about 20 kHz to about 21 kHz.

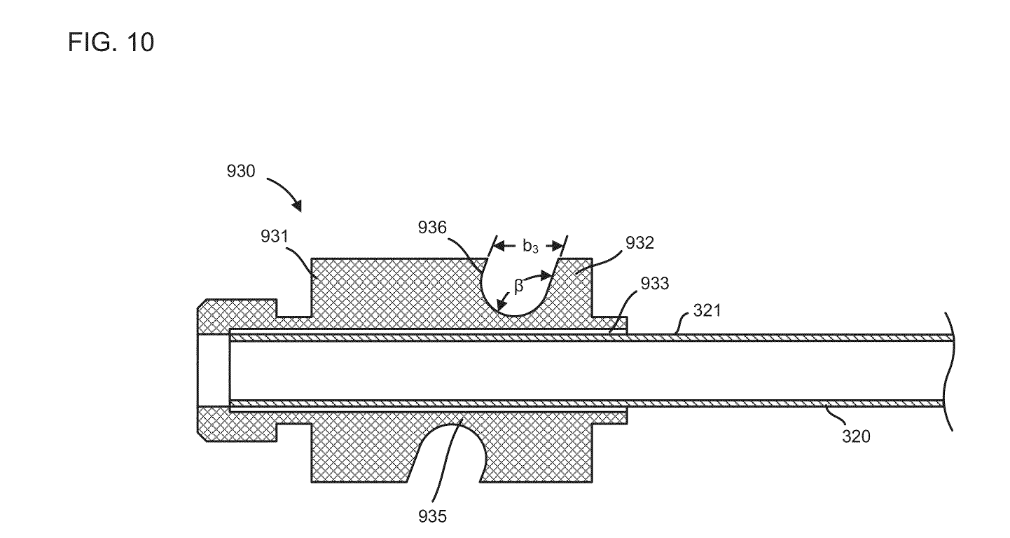

In some embodiments, a coupler can include one or more curved cut helical grooves on a surface of the coupler, configured to transform at least a portion of a linear component of an ultrasonic vibration produced by an ultrasonic energy source into a torsional vibration within a transmission member. For example, FIG. 10 shows a cross section of a coupler 930. The coupler 930 includes a first portion 931, a second portion 932, and a third portion 935 disposed between the first portion 931 and the second portion 932. The coupler 930 defines a passageway 933 configured to fixedly receive a proximal end portion 321 of a transmission member 320, for example, a flexible transmission member, or any other transmission member described herein. The first portion 931 is configured to be coupled to an ultrasonic energy source, for example, the transducer assembly 150 shown and described with respect to FIG. 2. For example, the first portion 931 can include a threaded portion configured to form a coupling with a transducer horn, for example transducer horn 163, as described above in detail with reference to FIG. 2. The coupler 930 can be configured to transfer at least a portion of an ultrasonic vibration produced by the ultrasonic energy source to the transmission member. The ultrasonic vibration produced by the ultrasonic energy source can be a linear vibration, i.e., includes a linear component.

The third portion 935 defines a curved cut helical groove 936. Thus, an outer surface of the first portion 931 can be discontinuous from the outer surface of the second portion 932 and the third portion 935. The groove 936 defines a width b₃ and a cut angle α. In some embodiments, the width b₃ can be in the range of about 0.04 inch to about 1.97 inch. In some embodiments, the cut angle α can be in the range of about 0 degrees to about 180 degrees. As shown in FIG. 10, the coupler 930 includes a single curved cut helical groove. In some embodiments, the coupler 930 can include a set of curved cut helical grooves, e.g., 2, 3, 4, 5, or even more. The groove 936 can sized such that the first portion 931, the second portion 932 and the third portion 935 are collectively configured to transform at least a portion of the linear component of the ultrasonic vibration into a torsional component within the transmission member (e.g., a flexible transmission member), as described in detail with respect to the coupler 830.

Furthermore, the width b₃ and the cut angle α can be varied such that the first portion 931, the second portion 932, and the third portion 935 are collectively configured to adjust a resonant frequency of the transmission member 320, or otherwise a probe assembly that includes the transmission member 320 to correspond to a vibrational frequency of the ultrasonic vibration produced by the ultrasonic energy source. For example, the first portion 931, the second portion 932, and the third portion 935 can be collectively configured to adjust the resonant frequency of the transmission member 320, or otherwise a probe assembly that includes the transmission member 320 to be in the range of about 20 kHz to about 21 kHz.

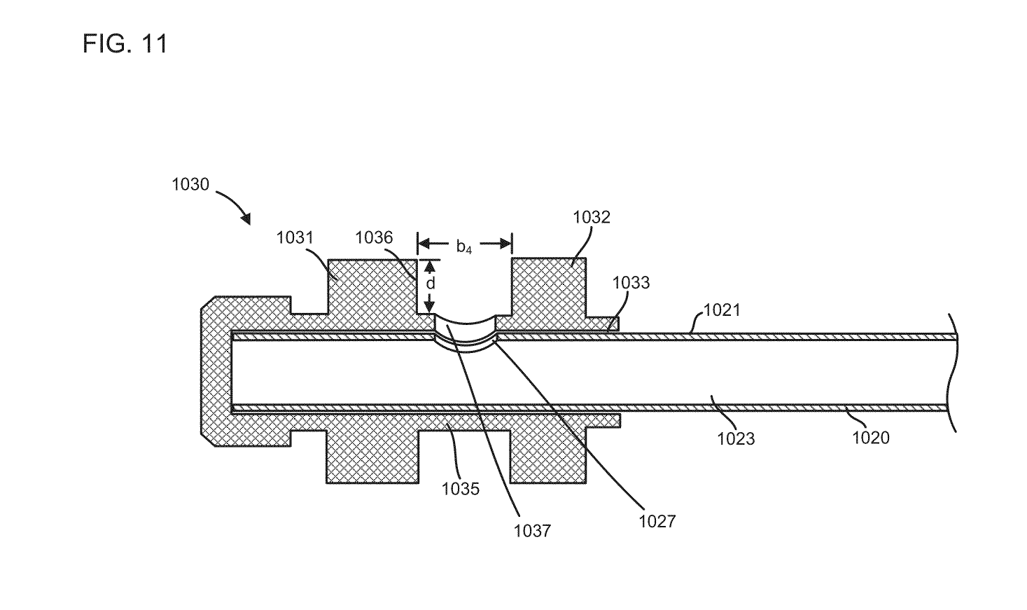

In some embodiments, a coupler can include a lateral opening in fluidic communication with the passageway which can be positioned to be in fluidic communication with a lumen (e.g., an irrigation lumen) of a transmission member. For example, FIG. 11 shows a cross section of a coupler 1030. The coupler 1030 includes a first portion 1031, a second portion 1032, and a third portion 1035 disposed between the first portion 1031 and the second portion 1032. The coupler defines a passageway 1033 configured to fixedly receive a proximal end portion 1021 of a transmission member 1020, for example, a flexible transmission member. The proximal end portion 1021 of the transmission member 1020 includes an opening 1027 defined on a side wall of the proximal end portion 1021 of the transmission member 1020. The opening 1027 is in fluidic communication with a lumen 1023, for example, an irrigation lumen defined by the transmission member 1020. The first portion 1031 is configured to be coupled to an ultrasonic energy source, for example, the transducer assembly 150 shown and described with respect to FIG. 2. For example, the first portion 1031 can include a threaded portion configured to form a coupling with a transducer horn, for example transducer horn 163, as described above in detail with reference to FIG. 2. The coupler 1030 can be configured to transfer at least a portion of an ultrasonic vibration produced by the ultrasonic energy source to the transmission member. The ultrasonic vibration produced by the ultrasonic energy source can be a linear vibration, i.e., includes a linear component.

The third portion 1035 defines a groove 1036. Thus, an outer surface of the first portion 1031 can be discontinuous from the outer surface of the second portion 1032 and the third portion 1035. The groove 1036 defines a width b₄ and a depth d. In some embodiments, the width b₄ can be about 0.1 inches, 0.11 inches, 0.12 inches, 0.13 inches, 0.14 inches, 0.15 inches, 0.16 inches, 0.17 inches, 0.18 inches, 0.19 inches, or about 0.20 inches, inclusive of all ranges therebetween. For example, in some embodiments, the width b₄ can be about 0.15 inches. The groove 1036 can be sized such that the first portion 1031, the second portion 1032 and the third portion 1035 are collectively configured to transform at least a portion of the linear component of the ultrasonic vibration into a torsional component within the transmission member (e.g., a flexible transmission member), as described in detail with respect to the coupler 830. Also, the width b₄ and the depth d can be varied such that the first portion 1031, the second portion 1032, and the third portion 1035 are collectively configured to adjust a resonant frequency of the transmission member 1020, or otherwise a probe assembly that includes the transmission member 1020 to correspond to a vibrational frequency of the ultrasonic vibration produced by the ultrasonic energy source (e.g., in the range of about 20 kHz to about 21 kHz).

Furthermore, the third portion 1035 defines a lateral opening 1037 at the base of the groove 1036. The lateral opening 1037 is in fluidic communication with the passageway 1033 defined by the coupler 1030. Moreover, the lateral opening 1037 is positioned to be in fluidic communication with the lumen 1023 defined by the transmission member 1020. For example, the proximal end portion 1021 of the transmission member 1020 can be disposed in the passageway 1033 and positioned such that the opening 1027 defined in the proximal end portion 1021 is substantially adjacent to the lateral opening 1037 defined by the coupler 1030. In this manner, the lateral opening 1037 of the coupler 1030 can be in fluidic communication with the lumen 1023 of the transmission member 1020. The lateral opening 1037 can, for example, enable delivery of an irrigation and/or aspirating fluid to the lumen 1023 of the transmission member 1021 from a separate conduit (not shown) coupled to the lateral opening 1037. In this manner, the fluid is not delivered through the ultrasonic energy source coupled to the coupler 1030, for example, the transducer assembly 150, thus simplifying manufacturing of the ultrasonic energy source.

The embodiments and/or components described herein can be packaged independently or any portion of the embodiments can be packaged together as a kit which can include any of the components described herein, for example, an ultrasonic generator (e.g., the ultrasonic generator 180), foot pedals (e.g., the foot pedals 170), one or more ultrasonic transducer assembly (e.g., the ultrasonic transducer assembly 150), one or more probe assemblies, for example, probe assemblies including a flexible transmission member, a semi-flexible transmission member, and/or a rigid transmission member, power cords, and any other auxiliary components or instruments.

In some embodiments, a kit can only include consumables to be used with an ultrasonic energy source, for example, the transducer assembly 150. For example, in some embodiments, a kit can include a first transmission member and a second transmission member, for example, the transmission member 130, 230, 330, 1030, or any other transmission member described herein. A proximal end portion of the first transmission member can be fixedly coupled to a first coupler, for example, the coupler 330, 430, 530, 630, 730, 830, 930, 1030, or any other coupler described herein. The first coupler defines a passageway in fluidic communication with an irrigation lumen of the first transmission member. The first coupler is configured to couple the first transmission member to an ultrasonic transducer assembly (e.g., the transducer assembly 150) to transfer at least a portion of a first ultrasonic vibration from the ultrasonic transducer assembly to the first transmission member such that the first transmission member and the first coupler, which can together form a first probe assembly, has a first resonant frequency. Similarly, a proximal end portion of the second transmission member can be fixedly coupled to a second coupler, for example, the coupler 330, 430, 530, 630, 730, 830, 930, 1030, or any other coupler described herein. The second coupler defines a passageway in fluidic communication with an irrigation lumen of the second transmission member. The second coupler is configured to couple the second transmission member to an ultrasonic transducer assembly (e.g., the transducer assembly 150) to transfer at least a portion of a second ultrasonic vibration from the ultrasonic transducer assembly to the second transmission member. Furthermore, the second transmission member and the second coupler, which can together form a second probe assembly, can have a second resonant frequency which is different from the first resonant frequency. Each of the first resonant frequency and the second resonant frequency can be in the range of about 20 kHz to about 21 kHz. For example, the first resonant frequency can be about 20.8 kHz, and the second frequency can be about 20.1 kHz.

In some embodiments, the first transmission member can define a first flexural stiffness, and the second transmission member can define a second flexural stiffness that is different than the first flexural stiffness. For example, the first transmission member can be semi-rigid transmission member and the second transmission member can be a rigid transmission member. Thus, the first coupler can be configured to adjust the first resonant frequency of the first transmission member coupled thereto, for example, the semi-rigid transmission member, and the second coupler can be configured to adjust the second resonant frequency of the second transmission member coupled thereto, for example, the rigid transmission member, such that the first resonant frequency is different from the second resonant frequency. This can be used to determine the flexural stiffness of the transmission member, for example, determine if a transmission member is the first transmission member (e.g., a semi-rigid transmission member) or the second transmission member (e.g., a rigid transmission member).

By way of example, in some embodiments, an ultrasonic transducer assembly, (e.g., the ultrasonic transducer assembly 150) configured to be coupled to the first transmission member or the second transmission member, or an ultrasonic generation in electric communication with the transducer assembly, can include a control module. The control module can be configured to detect the first resonant frequency and the second resonant frequency. Furthermore, the control module can be configured to produce a signal associated with (a) the first transmission member when the first transmission member is coupled to the ultrasonic transducer assembly or, (b) the second transmission member when the second transmission member is coupled to the ultrasonic transducer assembly.

Expanding further, the control module can include algorithms or hardware configured to detect a resonant frequency of a transmission member, for example, the first resonant frequency of the first transmission member or the second resonant frequency of the second transmission member coupled to the ultrasonic transducer assembly. For example, the transducer assembly can include a feedback mechanism, for example, vibration sensors, accelerometers, piezo detection elements, or any other electronic components configured to detect a resonant frequency of the transmission member coupled thereto (e.g., the first resonant frequency or the second resonant frequency). The resonant frequency signal detected can then be compared by the control module to the expected magnitude of the first resonant frequency and the second resonant frequency. If the magnitude of measured resonant frequency signal is substantially similar to the first resonant frequency, the control module can determine that the first transmission member is coupled to the ultrasonic energy source. The control module can then inform the user of the flexural stiffness of the transmission member. The informing can be any suitable alert, for example, an audio alert (e.g., a voice announcing which transmission member is coupled to the transducer assembly) or a visual alert (e.g., a message on a screen, illuminating light sources such as, for example, LED lights corresponding to the first transmission member or the second transmission member, etc.). In this manner, adjustment of the first resonant frequency of the first probe assembly by the first coupler, and the second resonant frequency of the second probe assembly by the second coupler can provide a facile mechanism for determining the flexural stiffness of the transmission member coupled to the transducer assembly. In some embodiments, the first transmission member and/or the second transmission member can be configured to deliver the first ultrasonic vibration and/or the second ultrasonic vibration to a kidney stone (or other tissue) sufficient to disintegrate the kidney stone (or tissue).

In some embodiments, the first coupler and/or the second coupler can include a first portion, a second portion, and a third portion disposed between the first portion and the second portion. The third portion can include a groove having a width such that the first portion, the second portion and the third portion are collectively configured to transform at least a portion of a linear component of the first ultrasonic vibration and/or the second ultrasonic vibration into a torsional component within the first transmission member. In such embodiment, the first coupler can include, for example, the coupler 730, 830, 930, or 1030, as described before herein.

In some embodiments, the kit can also include a third transmission member. A proximal end portion of the third transmission member can be fixedly coupled to a third coupler, for example, the coupler 330, 430, 530, 630, 730, 830, 930, 1030, or any other coupler described herein. The third coupler can define a passageway in fluidic communication with an irrigation lumen of the third transmission member. The third coupler is configured to couple the third transmission member to an ultrasonic transducer assembly (e.g., the transducer assembly 150) to transfer at least a portion of a third ultrasonic vibration from the ultrasonic transducer assembly to the third transmission member such that the third transmission member and the third coupler, which can together form a third probe assembly, have a third resonant frequency which is different from the first resonant frequency and the second resonant frequency. The third resonant frequency can be in the range of about 20 kHz to about 21 kHz. Furthermore, the third transmission member can include a third flexural stiffness different than the first flexural stiffness and the second flexural stiffness. Said another way, the kit can include three transmission members or otherwise three probe assemblies, each having a different flexural stiffness. For example, the first transmission member can be semi-flexible transmission member, the second transmission member can be rigid transmission member, and the third transmission member can be a flexible transmission member, as described herein. Furthermore, in such embodiments, the control module can be also be operative to detect the third resonant frequency and produce a signal associated with the third resonant frequency when the third transmission member is coupled to the ultrasonic transducer assembly. In this manner, the first, second or third transmission members can be used alternatively with the same ultrasonic energy source, for example, the ultrasonic transducer assembly 150 or any other transducer assembly described herein, depending upon the application. For example, the first transmission member (e.g., a semi-rigid transmission member) and the second transmission member (e.g., a rigid transmission member) can be used for delivering ultrasonic vibrations to kidney stones to disintegrated kidney stones. Moreover, the third transmission member (e.g., a flexible transmission member) can be used for delivering ultrasonic vibration to a target tissue within a vasculature of patient to disintegrate a target tissue, for example, a blood clot or cancer cell. In some embodiments, the third coupler can be configured to transform at least a portion of a linear component of the ultrasonic vibration received from the ultrasonic energy source into a torsional component within the third transmission member. In such embodiments, the third coupler can be any suitable coupler configured to transform at least a portion of the linear component of the vibration into a torsional component, for example, coupler 730, 830, 930, 1030 or any other coupler described herein that can perform the transformation. Each of the first, second, and third transmission members included in the kit can be one time use and disposable.

In some embodiments, a kit can also include non-consumables such as, for example, an ultrasonic energy generator, one or more transducer assemblies, and a set of transmission members having different flexural stiffnesses, for example, flexible, semi-flexible, and rigid transmission members. For example, in some embodiments, a kit can include a first ultrasonic transducer assembly and a second ultrasonic transducer assembly (e.g., such as the ultrasonic transducer assembly 150 described above with reference to FIG. 2), a flexible transmission member, a semi-flexible transmission member, and a rigid transmission member. The flexible transmission member has a first coupler coupled thereto, for example, the coupler member 430 or 530 described with reference to FIG. 5A-B and FIG. 6A-B, respectively. The first coupler is configured to couple the flexible transmission member to the first transducer assembly. The first coupler is further operative to match the vibrational mode and vibrational frequency of the flexible transmission member to the first transducer assembly, such that the transmitted vibrational frequency is in the range above 20 kHz (e.g., between about 20 kHz and about 21 kHz). Similarly, the semi-flexible transmission member and the rigid transmission member also include a second and a third coupler coupled thereto (e.g. coupler 630 described with reference to FIG. 7A-B), respectively. Each of the second coupler and the third coupler is configured to couple the semi-flexible transmission member to the first transducer assembly and the rigid transmission member to the second transducer assembly. Each of the second coupler and the third coupler are further operative to match the vibrational mode and vibrational frequency of the semi-flexible transmission and the rigid transmission member to the second transducer assembly, respectively such that the transmitted vibrational frequency is in the range of between about 20 kHz and about 21 kHz.

In some embodiments, a kit can include a single transducer assembly. In such embodiments, each of the flexible transmission member, the semi-flexible transmission member and the rigid transmission member includes a coupler (e.g., coupler 730, coupler 830, or any other coupler defined herein) coupled thereto. Each of the coupler is operative to match the vibrational mode and vibrational frequency of each of the flexible, semi-flexible and rigid transmission members to the transducer assembly to be in the range of about 20 kHz to about 21 kHz. For example, the flexible transmission member can be coupled to a first coupler configured such that the first flexible transmission member and the first coupler have a first resonant frequency. Moreover, the semi-flexible transmission member can be coupled to a second coupler configured such that the semi-flexible transmission member and the second coupler have a second resonant frequency. Furthermore, the rigid transmission member can be coupled to a third coupler configured such that the rigid transmission member and the third coupler have a third resonant frequency, the first resonant frequency, the second resonant frequency, and the third resonant frequency being different from each other.

In some embodiments, a kit can include an ultrasonic generator similar to the ultrasonic generator 180 shown and described above. The ultrasonic generator can be configured to distinguish each transmission member contained within the kit, and can automatically adjust the electronic signal produced and/or conveyed to the ultrasonic transducer assembly to correspond to the transmission member coupled thereto. For example, because transmission members defining different levels of flexural stiffness may also have different natural (or resonant) frequencies, in such embodiments, the ultrasonic generator can adjust the frequency of the electronic signal produced to correspond to the natural frequency of the transmission member that is coupled to the ultrasonic transducer assembly. In some embodiments, the ultrasonic generator or the transducer assembly can include a control module, for example, a processor, configured to detect the first resonant, the second resonant frequency, and the third frequency, as described herein, and produce a signal associated with first transmission member, the second transmission member, and the third transmission member corresponding to whichever transmission member is coupler to the transducer assembly. In this manner, the ultrasonic generator can automatically determine which transmission member is coupled to the transducer assembly.

The processor included in any of the ultrasonic generators can be a general-purpose processor (e.g., a central processing unit (CPU)) or other processor configured to execute one or more instructions stored in the memory. In some embodiments, the processor can alternatively be an application-specific integrated circuit (ASIC) or a field programmable gate array (FPGA). The processor can be configured to execute specific modules and/or sub-modules that can be, for example, hardware modules, software modules stored in the memory and executed in the processor, and/or any combination thereof. In some embodiments, the ultrasonic generator 180 can include a memory, for example, flash memory, one time programmable memory, a random access memory (RAM), a memory buffer, a hard drive, a read-only memory (ROM), an erasable programmable read-only memory (EPROM), and/or so forth. In some embodiments, the memory includes a set of instructions to cause the processor to execute modules, processes and/or functions used to generate, control, amplify, and/or transfer electric current to another portion of the system, for example, the transducer assembly 150.

Some embodiments described herein, such as, for example, embodiments related to the ultrasonic generators described above, relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using Java, C++, or other programming languages (e.g., object-oriented programming languages) and development tools. Additional examples of computer code include, but are not limited to, control signals, encrypted code, and compressed code.

The ultrasonic transmission members described herein can be fabricated and/or produced using any suitable methods. In some embodiments a transmission member can be formed via one of more manufacturing process. For example, in some embodiments, a transmission member can be formed via a tube drawing (e.g., drawn through a progressively smaller die (an extrusion process)).

Although certain transmission members (e.g., transmission member 320) are described above as being monolithically constructed, in other embodiments, any of the transmission members described herein can be constructed from two or more separately constructed components that are later joined together.

While the flexural stiffness of transmission members described above is spatially varied by altering the size or shape of the transmission member, in alternate embodiments, manufacturing techniques can be used to spatially vary of the flexural stiffness a transmission member while maintaining a uniform cross-sectional shape. For example, in some embodiments, a portion of a transmission member (e.g., the third portion 222 of the transmission member 220) can be heat-treated such that the elastic modulus of the portion of the transmission member is changed relative to the elastic modulus of a portion not heat treated. For example, in some embodiments, a portion of a transmission member can be tempered. In other embodiments, a transmission member in its entirety can be variably heat treated. For example, in some embodiments, a first portion can be tempered at a first temperature and a second portion can be tempered at a second temperature, different form the first. In this manner, the flexibility of the first portion and the flexibility of the second portion can be varied according to the temperature of tempering.

The transmission members described herein can be any suitable size. For example, in some embodiments, a transmission member (e.g., the transmission member 320) can have an outer diameter dₒ that is approximately 0.032 inches and an inner diameter dᵢ that is approximately 0.020 inches. In this manner, the transmission member can have a wall thickness of approximately 0.006 inches. In other embodiments, the outer diameter dₒ of the transmission member can be between approximately 0.014 to 0.050 inches and the inner diameter dᵢ can be between approximately 0.010 to 0.040 inches. In some embodiments, the working lengths of the of transmission member (e.g., transmission member 320) can range from about 15 inches to about 32 inches, inclusive of all ranges therebetween. For example, in some embodiments, a rigid transmission member can have an outer diameter dₒ of about 0.120 inches and working length of about 16 inches. In some embodiments, a semi-flexible transmission member can have an outer diameter dₒ of about 0.063 inches and a working length of about 22 inches. In some embodiments, a flexible transmission member can have an outer diameter dₒ of about 0.032 inches and a working length of about 32 inches.

In some embodiments, any of the flow tubes described herein, for example, the flow tube 157 can have an outer diameter of about 0.35 inches and an inner diameter of about 0.24 inches.

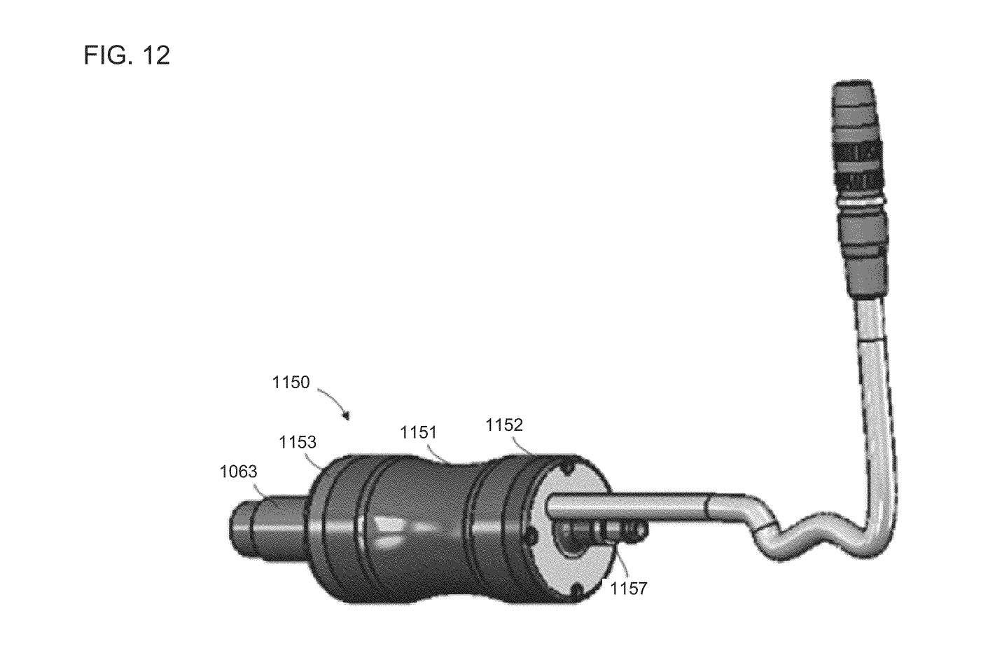

In some embodiments, an ultrasonic ablation system can include a transducer assembly configured to receive a flexible or semi-flexible transmission member. FIG. 12 shows a perspective view of a transducer assembly 1150 configured to receive a flexible transmission member, for example, any of the flexible transmission members described herein. The transmission member 1150 can be included in an ultrasonic ablation system, for example, the ultrasonic ablation system 100, or any other ultrasonic ablation system described herein. The transmission member 1150 can also be included in a kit, for example, any of the kits described herein. The transducer assembly 1150 has a proximal end portion 1152, a distal end portion 1153, a housing 1151, and a transducer horn 1163. The transducer horn 1163 is shaped and sized to be removably coupleable to a flexible transmission member, for example, a flexible transmission member that includes the coupler 430, 530, or any other flexible transmission member described herein. In some embodiments, the transducer horn 1163 can also include projections, features, any other coupling mechanisms and/or coupling members attached thereto, configured to couple the flexible transmission member to the transducer horn 1163. A flow tube 1157 is coupled to the distal end portion 1152 of the transducer assembly 1150. The transducer assembly 1150 further includes a ceramic ring, a back mass, a first electrode, a second electrode, a seal gasket, a socket head screw, an O-ring, a transducer cable, a back cover, an insulator tube, a front cover, a barb connector, an insulator ring, a flat screw and a stress screw.

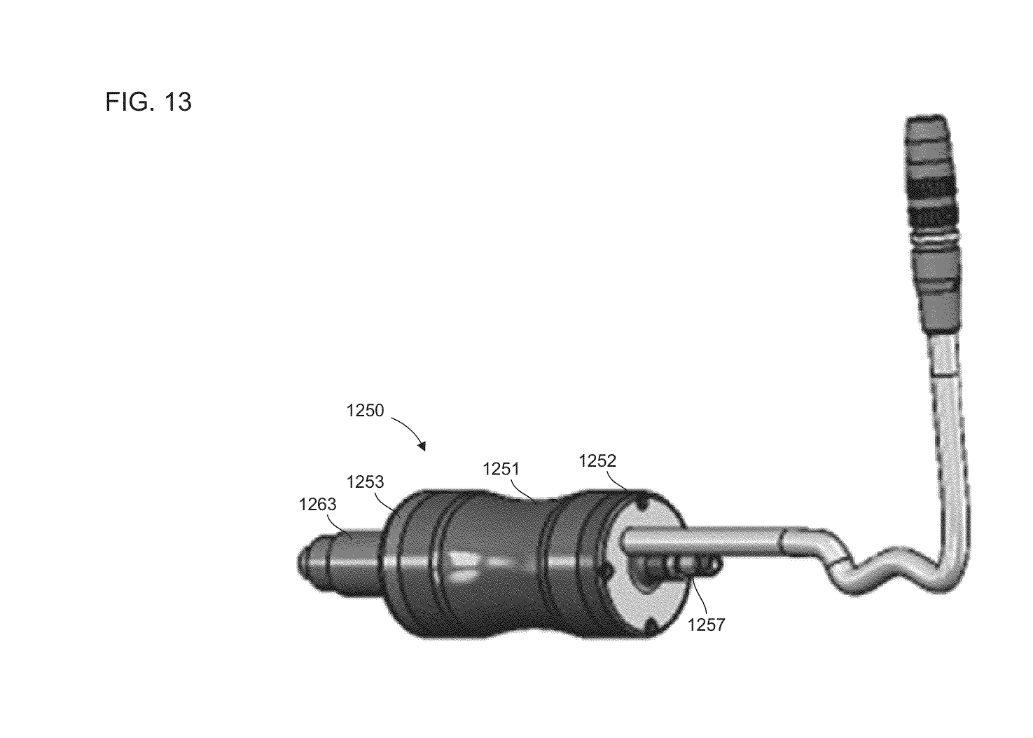

In some embodiments, an ultrasonic ablation system can include a transducer assembly configured to receive a rigid transmission member. FIG. 13 shows a perspective view of a transducer assembly 1250 configured to receive a semi-flexible and/or rigid transmission member, for example, any of the semi-flexible and rigid transmission members described herein. The transmission member 1250 can be included in an ultrasonic ablation system, for example, the ultrasonic ablation system 100, or any other ultrasonic ablation system described herein. The transmission member 1250 can also be included in a kit, for example, any of the kits described herein. The transducer assembly 1250 has a proximal end portion 1252, a distal end portion 1253, a housing 1251, and a transducer horn 1263. The transducer horn 1263 is shaped and sized to be removably coupleable to a semi-flexible and/or rigid transmission member, for example, a semi-flexible or rigid transmission member that includes the coupler 630, or any other semi-flexible or rigid transmission member described herein. In some embodiments, the transducer horn 1263 can also include projections, features and or any other coupling mechanisms or members attached thereto, configured to couple the semi-flexible transmission member and or the rigid transmission member to the transducer horn 1263. A flow tube 1257 is coupled to the distal end portion 1252 of the transducer assembly 1250. The transducer assembly 1250 further includes a ceramic ring, a back mass, a first electrode, a second electrode, a seal gasket, a socket head screw, an O-ring, a transducer cable, a back cover, an insulator tube, a front cover, a barb connector, an insulator ring, a flat screw and a stress screw.

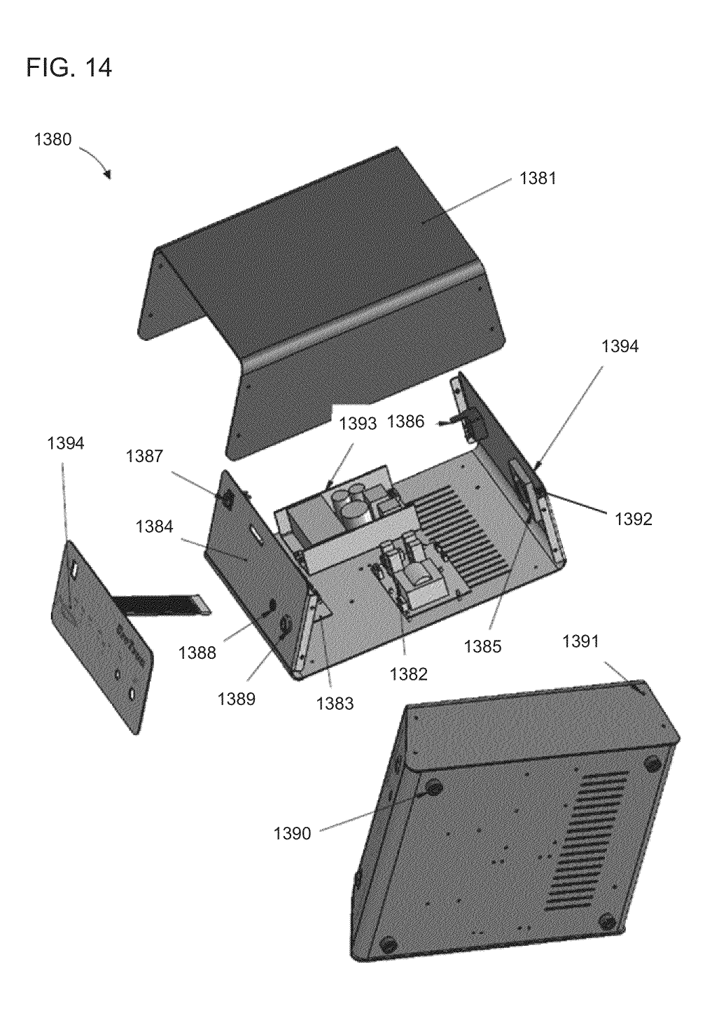

FIG. 14 shows an exploded view of an ultrasonic generator 1380 according to an embodiment which can be included in an ultrasonic ablation system, for example, ultrasonic ablation system 100, or any other ultrasonic ablation system described herein. The ultrasonic generator 1380 can also be included in a kit, for example any of the kits described herein. The ultrasonic generator 1380 includes a cover 1381, a driver board 1382, a control board 1383, a chassis 1384 that includes a nut, a fan 1385, a power entry 1386, a power switch 1387, a footswitch receptacle 1388, a transducer receptacle 1389, a round bumper 1390, a pan head screw 1391, a fan guard 1392, a power supply 1393 and a decal 1394.

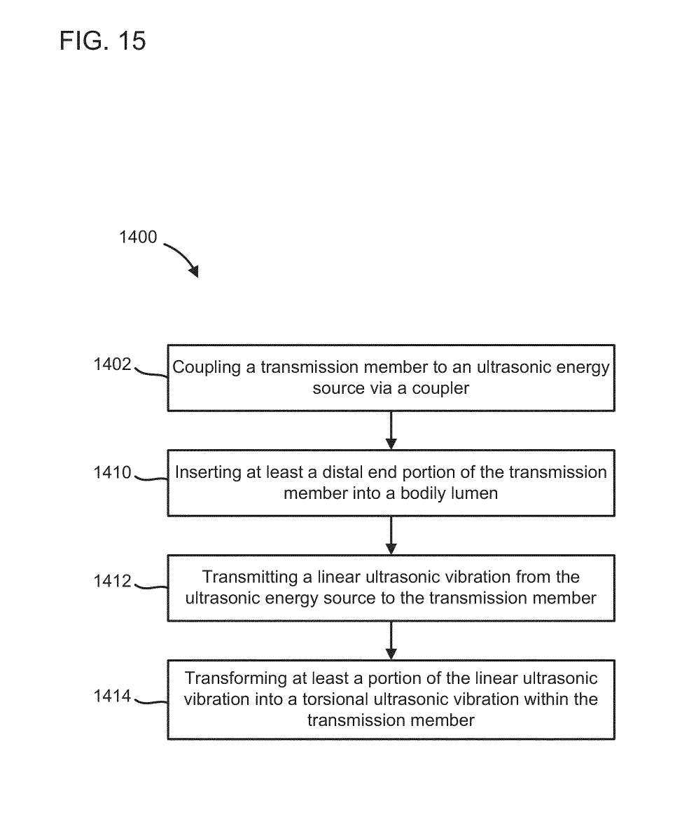

FIG. 15 shows a schematic flow diagram of a method 1400 for delivering a torsional ultrasonic vibration to a target tissue using a probe assembly that includes a transmission member and a coupler. The method 1400 includes coupling a transmission member to an ultrasonic energy source via a coupler, at 1402. The transmission member can include the transmission member 120, 320, 1020, or any other transmission member described herein. Furthermore, the transmission member can have any suitable flexural stiffness, i.e., the transmission member can be a flexible transmission member, a semi-flexible transmission member, or a rigid transmission member. A proximal end portion of the transmission member can be coupled to the coupler, for example, fixedly disposed in a passageway defined by the coupler. The coupler can be any coupler described herein which is configured to transform at least a portion of a linear component of an ultrasonic vibration into a torsional component within the transmission member, for example, the coupler 730, 830, 930, or 1030, or any other coupler configured to perform the transforming as described herein. The ultrasonic energy source can include the transducer assembly 150 or any other transducer assembly described herein, which is configured to produce an ultrasonic vibration that includes a linear component. Furthermore, the transmission member, the coupler and the ultrasonic energy source can be included in an ultrasonic energy ablation system, for example, the system 100.

A distal end portion of the transmission member is inserted into a bodily lumen, at 1410. The bodily lumen can include the vasculature, the urethra, the colon, a body cavity, or any other suitable bodily lumen. A linear ultrasonic vibration is transmitted from the ultrasonic energy source to the transmission member, at 1412. For example, the ultrasonic energy source can produce an ultrasonic vibration at least a portion of which includes a linear component. The linear component of the ultrasonic vibration can be communicated to the coupler and transmitted to the transmission member therefrom. At least a portion of the linear ultrasonic vibration is transformed into a torsional ultrasonic vibration within the transmission member, at 1414. For example, the coupler can include features, for example, one or more straight grooves, straight cut helical grooves, or curved cut helical grooves, as described with respect to the couplers 730, 830, and 930, respectively, configured to transform at least a portion of the linear ultrasonic vibration into a torsional component within the transmission member. In some embodiments, substantially all of the linear component of the ultrasonic vibration can be transformed into the torsional component within the transmission member such that the ultrasonic vibration delivered by the transmission member to the target tissue is substantially composed of the torsional component. In some embodiments, only a portion of the linear component is transformed into the torsional component such that the ultrasonic vibration delivered by the transmission member to the target tissue includes both a linear component and a torsional component (i.e., a bimodal vibration). Such a combination of the linear component and the torsional component of the ultrasonic vibration can, for example, be beneficial for ultrasonic ablation therapy within a vasculature of a patient, for example, for breaking clots, cancer cells, adipose tissue, etc.

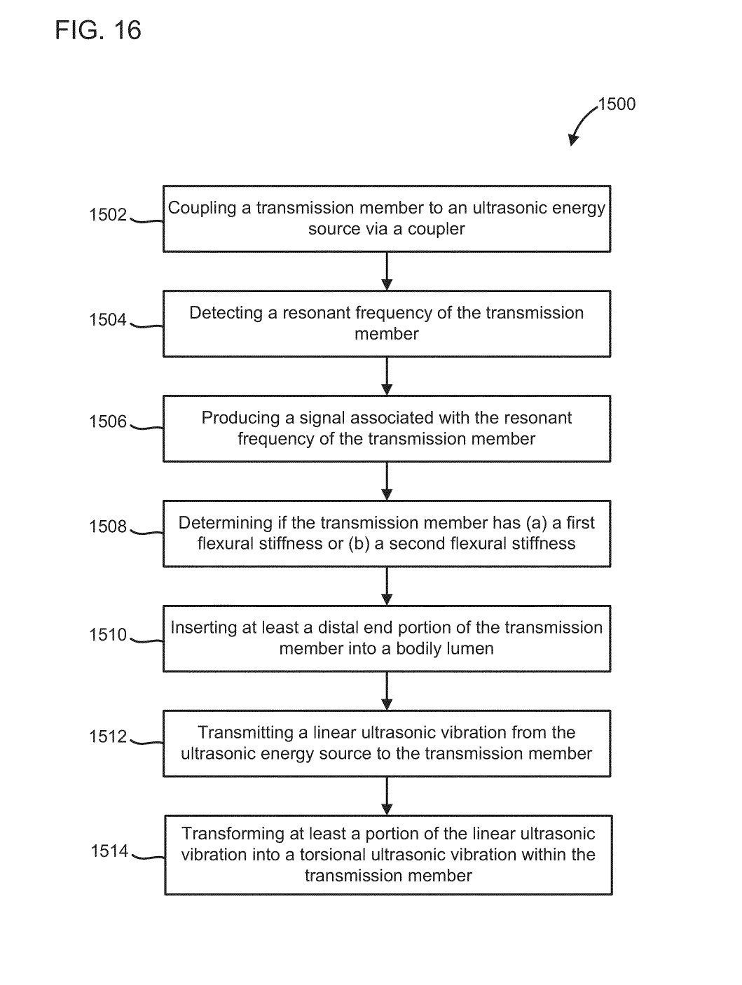

FIG. 16 shows a schematic flow diagram of a method 1500 for determining the flexural stiffness of a transmission member coupled to an ultrasonic energy source and delivering a torsional ultrasonic vibration to a target tissue using a probe assembly that includes a transmission member and a coupler. The method 1500 includes coupling a transmission member to an ultrasonic energy source via a coupler, at 1502. The transmission member can include the transmission member 120, 320, 1020, or any other transmission member described herein. Furthermore, the transmission member can have any suitable flexural stiffness, i.e., the transmission member can be a flexible transmission member, a semi-flexible transmission member, or a rigid transmission member. A proximal end portion of the transmission member can be coupled to the coupler, for example, fixedly disposed in a passageway defined by the coupler. The coupler can be any coupler described herein which is configured to transform at least a portion of a linear component of an ultrasonic vibration into a torsional component within the transmission member, for example, the coupler 730, 830, 930, or 1030, or any other coupler configured to perform the transforming as described herein. The ultrasonic energy source can include the transducer assembly 150 or any other transducer assembly described herein, which is configured to produce an ultrasonic vibration that includes a linear component. Furthermore, the transmission member, the coupler and the ultrasonic energy source can be included in an ultrasonic energy ablation system, for example, the system 100.

A resonant frequency of the transmission member is detected, at 1504. For example, the transmission member can have a first flexural stiffness (e.g., a semi-rigid transmission member) or a second flexural stiffness (e.g., a rigid transmission member). Furthermore, the transmission member or otherwise a probe assembly that includes the transmission member and the coupler can be configured to have a resonant frequency which varies based on the flexural stiffness of the transmission member. For example, if the transmission member has a first flexural stiffness (e.g., a semi-rigid transmission member), the coupler can be configured such that the coupler and the transmission member can be configured to have a first resonant frequency, for example, about 20.8 kHz. Moreover, if the transmission member has a second flexural stiffness (e.g., a rigid transmission member), the coupler can be configured such that the coupler and the transmission member have a second resonant frequency different from the first resonant frequency, for example, about 20.1 kHz. In this manner, the coupler can be configured to deliberately adjust the first resonant frequency to a first predetermined value and the second resonant value to a second predetermined value. The difference between the first and second resonant frequencies can be sufficient to allow detection and differentiation between the first resonant frequency and the second resonant frequency. The ultrasonic energy source can include hardware and software such as, for example, accelerometers, vibration sensors, piezo elements, or any other suitable components configured to detect the resonant frequency of the transmission member.

A signal is produced associated with the resonant frequency of the transmission member, at 1506. The detected resonant frequency can, for example, be transformed into a digital signal which can be communicated to a control module, for example, a processor included in the ultrasonic energy source (e.g., in a transducer assembly such as, for example, the transducer assembly 150, or an ultrasonic generator such as, for example, the ultrasonic generator 180). The digital signal can be configured to correspond to the resonant frequency of the transmission member and the coupler. For example, a first signal can be produced if the transmission member and the coupler have a first resonant frequency (e.g., associated with the semi-flexible transmission member) and a second signal different from the first signal can be produced if the transmission member and the coupler have a second resonant frequency (e.g., associated with a rigid transmission member).

The method further includes determining if the transmission member has (a) a first flexural stiffness, or (b) a second flexural stiffness, at 1508. The control module can, for example, included algorithms configured to analyze the signal associated with the resonant frequency of the coupler and the transmission member and determine the flexural stiffness of the transmission member. For example, the control module can be configured to detect the first signal and associate the first signal with the first resonant frequency and thereby, the first flexural stiffness (e.g., associated with the semi-flexible transmission member). Similarly, the control module can detect the second signal and associate the second signal with the second resonant frequency and thereby, the second flexural stiffness (e.g., associated with the rigid transmission member). In this manner, the ultrasonic energy source can determine the flexural stiffness of the transmission member simply by coupling the transmission member to the ultrasonic energy source.

A distal end portion of the transmission member is inserted into a bodily lumen, at 1510. The bodily lumen can include the vasculature, the urethra, the colon, a body cavity, or any other suitable bodily lumen. A linear ultrasonic vibration is transmitted from the ultrasonic energy source to the transmission member, at 1512. For example, the ultrasonic energy source can produce an ultrasonic vibration at least a portion of which includes a linear component. The linear component of the ultrasonic vibration can be communicated to the coupler and transmitted to the transmission member therefrom. At least a portion of the linear ultrasonic vibration is transformed into a torsional ultrasonic vibration within the transmission member, at 1514. For example, the coupler can include features, for example, one or more straight grooves, straight cut helical grooves, or curved cut helical grooves, as described with respect to the couplers 730, 830, and 930, respectively, configured to transform at least a portion of the linear ultrasonic vibration into a torsional component within the transmission member. In some embodiments, substantially all of the linear ultrasonic component can be transformed into the torsional component within the transmission member such that the ultrasonic vibration delivered by the transmission member to the target tissue is substantially composed of the torsional component. In some embodiments, only a portion of the linear component is transformed into the torsional component such that the ultrasonic vibration delivered by the transmission member to the target tissue includes both a linear component and a torsional component (i.e., a bimodal vibration). Such a combination of the linear component and the torsional component of the ultrasonic vibration can, for example, be beneficial for ultrasonic ablation therapy within a vasculature of a patient, for example, for breaking clots, cancer cells, adipose tissue, etc.

Any of the couplers described herein can be formed from a substantially strong and rigid material such as, for example, aluminum, stainless steel, reinforced steel, nitinol, brass, copper, other metals or alloys, plastics, TEFLON®, polymers, carbon fiber, any other suitable material or combination thereof.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. Additionally certain events may be performed concurrently in parallel processes when possible, as well as performed sequentially. While the embodiments have been particularly shown and described, it will be understood that various changes in form and details may be made.

Although the transducer assembly 150 is shown in FIG. 2 as including two insulators 161 and two piezoelectric rings 162, in other embodiments, a transducer assembly can include any suitable number of insulators 161 and/or piezoelectric rings 162 in any suitable arrangement. Moreover, the insulators 161 can be formed from any suitable insulating material, ceramic materials (e.g., polyamide, expanded polytetrafluoroethylene (EPTFE), or the like). Similarly, the piezoelectric rings 162 can be any suitable piezoelectric material (e.g., lead zirkonate titanate (PZT-5), PZT-8, lead titanate (PT), lead metaniobate (PbNbO₆), polyvinylidenefluoride (PVDF), or the like).

Although various embodiments have been described as having particular features and/or combinations of components, other embodiments are possible having a combination of any features and/or components from any of embodiments where appropriate. For example, in some embodiments, a coupler can include a first portion, a second portion, and a third portion disposed between the first portion and the second portion and including a groove as described with respect to coupler 730 shown in FIG. 8. The coupler can be configured to transform at least a portion of a linear component of an ultrasonic vibration produced by an ultrasonic energy source into a torsional component within a transmission member, as described before herein. Furthermore, a first ratio between a length of the first portion and a length of a second portion and/or a second ratio between a length of the first portion and a length of the third portion can be varied to adjust a resonant frequency of the coupler and the transmission member coupled thereto to correspond to a vibrational frequency of the ultrasonic vibration produced by the ultrasonic energy source.

1. An apparatus comprising:
a coupler including a first portion and a second portion, the coupler defining a passageway configured to fixedly receive a proximal end portion of a transmission member, the first portion configured to be coupled to an ultrasonic energy source, the coupler configured to transfer at least a portion of an ultrasonic vibration produced by the ultrasonic energy source to the transmission member, the first portion and the second portion collectively configured to adjust a resonant frequency of the transmission member to correspond to a vibrational frequency of the ultrasonic vibration produced by the ultrasonic energy source.

2. The apparatus of claim 1, wherein the first portion has a first diameter and a first length, and the second portion has a second diameter and a second length, the first diameter greater than the second diameter, a ratio of the first length and the second length being such that the resonant frequency of the transmission member is in the range of about 20 kHz to about 21 kHz.

3. The apparatus of claim 1, wherein the transmission member is a semi-flexible transmission member.

4. The apparatus of claim 3, wherein the coupler is configured to adjust the resonant frequency of the semi-flexible transmission member to be about 20.9 kHz.

5. The apparatus of claim 2, wherein the coupler includes a third portion disposed between the first portion and the second portion, the third portion having a third diameter and a third length, the third diameter less than the first diameter and greater than the second diameter, a ratio of the first length, the second length, and the third length being such that the resonant frequency of the transmission member is in the range of about 20 kHz to about 21 kHz.

6. The apparatus of claim 1, wherein an outer surface of the first portion is discontinuous from an outer surface of the second portion.

7. The apparatus of claim 1, wherein:
the portion of the ultrasonic vibration includes a linear component; and
the first portion and the second portion collectively configured to transform at least a portion of the linear component of the ultrasonic vibration into a torsional component within the transmission member.

8. The apparatus of claim 1, wherein:
the first portion has a first diameter and a first length;
the second portion has a second diameter and a second length; and
the coupler includes a third portion disposed between the first portion and the second portion, the third portion having a third diameter and a third length, the first diameter greater than the second diameter and the third diameter, the second diameter less than the third diameter and greater than the second diameter,
a ratio of the first length and the second length being about 2.35, a ratio of the first length and the third length being about 0.61.

9. The apparatus of claim 5, wherein the ratio of the first length and the second length is about 1, and the ratio of the first length and the third length is about 0.83.

10. The apparatus of claim 1, wherein the coupler includes a third portion disposed between the first portion and the second portion, the third portion defining a groove, the first portion, the second portion and the third portion collectively configured to transform at least a portion of a linear component of the ultrasonic vibration into a torsional component within the transmission member.

11. The apparatus of claim 10, wherein the groove is a circumferential groove having a width and a depth configured to produce the torsional vibratory force.

12. The apparatus of claim 10, wherein the groove is a helical groove having an angular width and a cut angle, the helical groove configured to produce the torsional vibratory force.

13. The apparatus of claim 12, wherein the helical groove is at least one of a straight cut helical groove and a curved cut helical groove.

14. The apparatus of claim 1, wherein the coupler defines a lateral opening in fluidic communication with the passageway, the lateral opening positioned to be in fluidic communication with a lumen defined by the transmission member.

15. An apparatus comprising:
a coupler including a first portion and a second portion, the coupler defining a passageway configured to fixedly receive a proximal end portion of a transmission member, the first portion configured to be coupled to an ultrasonic energy source, the coupler configured to transfer at least a portion of an ultrasonic vibration produced by the ultrasonic energy source to the transmission member, the portion of the ultrasonic vibration includes a linear component, the first portion and the second portion collectively configured to transform at least a portion of the linear component of the ultrasonic vibration into a torsional component within the transmission member.

16. The apparatus of claim 15, wherein an outer surface of the first portion is discontinuous from an outer surface of the second portion.

17. The apparatus of claim 15, wherein the coupler includes a third portion disposed between the first portion and the second portion, the third portion defining a groove, the first portion, the second portion and the third portion collectively configured to produce the torsional component within the transmission member.

18. The apparatus of claim 17, wherein the groove is a circumferential groove having a width and a depth configured to produce the torsional vibratory force.

19. The apparatus of claim 17, wherein the groove is a helical groove having an angular width and a cut angle, the helical groove configured to produce the torsional vibratory force.

20. The apparatus of claim 19, wherein the helical groove is at least one of a straight cut helical groove and a curved cut helical groove.

21. A kit, comprising:
a first transmission member, a proximal end portion of the first transmission member fixedly coupled to a first coupler, the first coupler defining a passageway in fluid communication with an irrigation lumen of the first transmission member, the first coupler configured to couple the first transmission member to an ultrasonic transducer assembly to transfer at least a portion of a first ultrasonic vibration from the ultrasonic transducer assembly to the first transmission member, the first coupler configured to such that the first transmission member and the first coupler have a first resonant frequency; and
a second transmission member, a proximal end portion of the second transmission member fixedly coupled to a second coupler, the second coupler defining a passageway in fluid communication with an irrigation lumen of the second transmission member, the second coupler configured to couple the second transmission member to the ultrasonic transducer assembly to transfer at least a portion of a second ultrasonic vibration from the ultrasonic transducer assembly to the second transmission member, the second coupler configured to such that the second transmission member and the second coupler have a second resonant frequency, the second resonant frequency different from the first resonant frequency.

22. The kit of claim 21, wherein the first resonant frequency and the second resonant frequency are configured to be in the range of about 20 kHz to about 21 kHz.

23. The kit of claim 21, wherein the first resonant frequency is about 20.8 kHz and the second resonant frequency is about 20.1 kHz.

24. The kit of claim 21, wherein:
the first transmission member defines a first flexural stiffness; and
the second transmission member defines a second flexural stiffness, the second flexural stiffness different than the first flexural stiffness.

25. The kit of claim 22, wherein the first coupler includes a first portion, a second portion and a third portion, the third portion disposed between the first portion and the second portion, the third portion including a groove having a width, the first portion, the second portion and the third portion collectively configured to transform at least a portion of a linear component of the first ultrasonic vibration into a torsional component within the first transmission member.

26. The kit of claim 24, further comprising:
a third transmission member, a proximal end portion of the third transmission member fixedly coupled to a third coupler, the third coupler defining a passageway in fluid communication with an irrigation lumen of the third transmission member, the third coupler configured to couple the third transmission member to the ultrasonic transducer assembly to transfer at least a portion of a third ultrasonic vibration from the ultrasonic transducer assembly to the third transmission member, the third coupler configured to such that the third transmission member and the third coupler have a third resonant frequency, the third resonant frequency different from the first resonant frequency and the second resonant frequency.

27. The kit of claim 26, wherein the third transmission member has a third flexural stiffness, the third flexural stiffness different than the first flexural stiffness and the second flexural stiffness.

28. The kit of claim 21, further comprising:
the ultrasonic transducer assembly including a control module configured to detect the first resonant frequency and the second resonant frequency, and produce a signal associated with (a) the first transmission member when the first transmission member is coupled to the ultrasonic transducer assembly or, (b) the second transmission member when the second transmission member is coupled to the ultrasonic transducer assembly.

29. The kit of claim 24, wherein the at least one of the first transmission member and the second transmission member are configured to deliver the ultrasonic vibration to a kidney stone, the ultrasonic vibration configured to disintegrate the kidney stone.

30. A method comprising:
coupling a transmission member to an ultrasonic energy source via a coupler, a proximal end portion of the transmission member fixedly coupled to the coupler;
inserting at least a distal end portion of the transmission member into a bodily lumen;
transmitting a linear ultrasonic vibration from the ultrasonic energy source to the transmission member; and
transforming at least a portion of the linear ultrasonic vibration into a torsional ultrasonic vibration within the transmission member.

31. The method of claim 30, further comprising:
detecting a resonant frequency of the transmission member;
producing a signal associated with the resonant frequency of the transmission member; and
determining if the transmission member is (a) a first flexural stiffness or (b) a second flexural stiffness.

32. The method of claim 31, wherein the coupler includes a first portion, a second portion, and a third portion, the third portion defining a groove, the first portion, the second portion and the third portion collectively configured to transform at least a portion of the linear ultrasonic vibration into the torsional ultrasonic vibration within the transmission member.
